(19) United States
(12) Patent Application Publication
(10) Pub. No.: US 2024/0139167 A1
(43) Pub. Date: May 2, 2024

(54) LYSOSOME-ASSOCIATED MEMBRANE PROTEIN TARGETING COMPOUNDS AND USES THEREOF

(71) Applicants: HADASIT MEDICAL RESEARCH SERVICES AND DEVELOPMENT LTD., Jerusalem (IL); RAMOT AT TEL-AVIV UNIVERSITY LTD., Tel Aviv (IL)

(72) Inventors: Or KAKHLON, Jerusalem (IL); Miguel Enrique WEIL, Tel Aviv (IL)

(21) Appl. No.: 18/277,257

(22) PCT Filed: Feb. 16, 2022
(86) PCT No.: PCT/IL2022/050187

§ 371 Date: Aug. 15, 2023

Related U.S. Application Data
(60) Provisional application No. 63/149,730, filed on Feb. 16, 2021.

(51) Int. Cl. A61K 31/4436; A61P 3/00

(52) U.S. Cl. CPC A61K 31/4436; A61P 3/00

(57) ABSTRACT

Disclosed herein is a polypeptide including a pharmaceutical composition for use in prevention or treatment of a disease or a disorder associated with lysosomal storage and an autophagy-misregulation associated disease. Further provided are agents that bind a region of an N-terminal domain of a lysosomal-associated membrane protein 1 (LAMP-1), and methods for treating or preventing development of a disease or a disorder associated with lysosomal storage, polyglucosan accumulation or abnormal glycogen accumulation and autophagy-misregulation in a subject in need thereof.

### CROSS REFERENCE TO RELATED APPLICATIONS

This application claims the benefit of priority of U.S. Provisional Patent Application No. 63/149,730, filed Feb. 16, 2021, the contents of which are all incorporated herein by reference in their entirety.

### FIELD OF THE INVENTION

The present invention is in the field of preventing and treating certain diseases or disorders associated with lysosomal storage, polyglucosan accumulation or abnormal glycogen accumulation, abnormal protein accumulations, and autophagy-misregulation associated diseases as well as in the field of screening for agents that prevent and treat these diseases.

### BACKGROUND OF THE INVENTION

Lysosomes are subcellular organelles responsible for the physiologic turnover of cell constituents. They contain catabolic enzymes, which require a low pH environment in order to function optimally. Lysosomal storage diseases (LSD) describe a heterogeneous group of dozens of rare inherited disorders characterized by the accumulation of undigested or partially digested macromolecules, which ultimately results in cellular dysfunction and clinical abnormalities. LSDs result from gene mutations in one or more lysosomal enzymes, resulting in accumulation of the enzyme substrates in lysosomes. Organomegaly, connective-tissue and ocular pathology, and central nervous system dysfunction may result.

Neurological impairment and neurodegenerative processes are associated with lysosomal dysfunction and represent a predominant feature in most LSDs. Neuropathology can occur in multiple brain regions (e.g., thalamus, cortex, hippocampus, and cerebellum) and involves unique temporal and spatial changes, which often entail early region-specific neurodegeneration and inflammation. As an example, Purkinje neurons degenerate in many of these diseases leading to cerebellar ataxia.

Glycogen is a branched polysaccharide with a molecular weight of nine to ten million daltons. The average glycogen molecule contains about 55,000 glucose residues linked by α-1,4 (92%) and α-1,6 (8%) glycosidic bonds. The synthesis of glycogen is catalyzed by two enzymes: (i) glycogen synthase, which “strings” glucose to form linear chains; and (ii) the glycogen branching enzyme (GBE), which attaches a new short branch of glucose units to a linear chain in an α-1,6 glycosidic bond. Glycogen is stored primarily in liver and muscle, where it represents an energy reserve that can be quickly mobilized. The most common disorder of glycogen metabolism is seen in diabetes, in which abnormal amount of insulin or abnormal insulin response result in accumulation or depletion of liver glycogen. Although glycogen synthesis and breakdown have been studied for decades, their control is not completely understood.

Adult Polyglucosan Body Disease (APBD), is a glycogen storage disorder (GSD) which manifests as a debilitating and fatal progressive axonopathic leukodystrophy from the age of 45-50. APBD is further characterized by peripheral neuropathy, dysautonomia, urinary incontinence and occasionally dementia, all being important diagnostic criteria for this commonly misdiagnosed and widely heterogeneous disease. APBD is caused by glycogen branching enzyme (GBE) deficiency leading to poorly branched and therefore insoluble glycogen (polyglucosans, PG), which precipitate, aggregate and accumulate into PG bodies (PB). Being out of solution and aggregated, PB cannot be digested by glycogen phosphorylase. The amassing aggregates lead to liver failure and death in childhood (Andersen's disease; GSD type IV). Milder mutations of GBE, such as p.Y329S in APBD, lead to smaller PB, which do not disturb hepatocytes and most other cell types, merely accumulating in the sides of cells. In neurons and astrocytes, however, over time PB plug the tight confines of axons and processes and lead to APBD.

While an effective cure for APBD is currently missing and is urgently needed, APBD represents the larger group of GSDs. GSDs are a versatile group of 15 incurable diseases with a combined frequency of 1 in 20,000-43,000. Ranging from child liver disorders such as GSD1, through adolescent myoclonic epilepsies such as the Lafora Disease (LD), and adult progressive neurodegenerative disorders such as APBD, all GSDs are currently incurable. There is still a need for therapies, agents, and improved and correlative diagnostics for lysosomal storage diseases, and glycogen storage disorders.

### SUMMARY OF THE INVENTION

In one aspect of the invention, there is provided a pharmaceutical composition for use in prevention or treatment of a disease or a disorder selected from a lysosomal storage associated disease and an autophagy-misregulation associated disease, the pharmaceutical composition comprising a compound, pharmaceutically acceptable salt, isomer or tautomer thereof, wherein the compound is represented by Formula I:

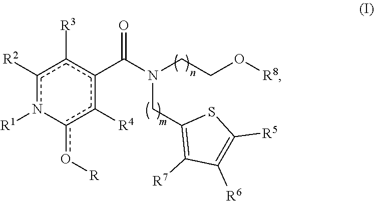

wherein:

[symbol] represents a single or a double bond;
n and m each independently represents an integer in a range from 1 to 3;
R and R¹ each independently represents hydrogen, or is absent; and
R³, R⁴, R⁵, R⁶, R⁷ and R⁸ each independently represents hydrogen, or is selected from the group comprising alkyl, cycloalkyl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, substituted or non-substituted.

In some embodiments, n and m is 1.

In some embodiments, R², R⁷ and R⁸ represent a methyl.

In some embodiments, the compound is selected from:

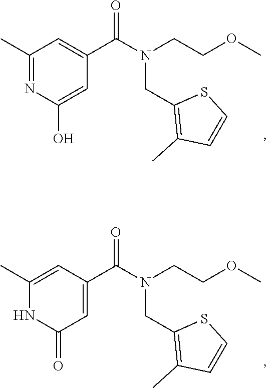

or both.

In some embodiments, the lysosomal storage associated disease is selected from the group consisting of: Gaucher disease, Fabry disease, Tay-Sachs disease, Mucopolysaccharidoses (MPS) diseases, aspartylglucosaminuria, GM1-gangliosidosis, Krabbe (globoid cell leukodystrophy or galactosylceramide lipidosis), Metachromatic, leukodystrophy, Sandhoff disease, mucolipidosis type II (I-cell disease), mucolipidosis type IIIA (pseudo-Hurler poly dystrophy), Niemann-Pick disease type C2 and C1, Danon disease, free sialic acid storage disorder, mucolipidosis type IV, and multiple sulfatase deficiency (MSD), metabolic disorders, obesity, type II diabetes and insulin resistance.

In some embodiments, the autophagy-misregulation associated disease is characterized by reduced or misregulated autophagic activity. In some embodiments, the autophagy-misregulation associated disease characterized by reduced or misregulated autophagic activity is selected from the group consisting of: Alzheimer's disease, and cancer associated with reduced autophagic activity.

In another aspect of the invention, there is provided a method for treating or preventing development of a disease or a disorder selected from a lysosomal storage associated disease and an autophagy-misregulation associated disease, in a subject in need thereof, comprising administering to the subject a therapeutically effective amount of the pharmaceutical composition of the present invention.

In another aspect of the invention, there is provided an agent that binds a region of an N-terminal domain of a lysosomal-associated membrane protein 1 (LAMP-1; SEQ ID NO: 1; FSVNYDTKSGPKNMTFDLPSDATVVLNRSSCGKENTSDPSLVIAFGRGHTLTLNF TRNATRYSV), wherein the region comprises any one of: SEQ ID NO: 2 (FSVNYD); and SEQ ID NO: 3 (NVTV).

In some embodiments, the agent inhibits a LAMP1:LAMP1 interaction.

In some embodiments, the agent is for use in prevention or treatment of a disease or a disorder associated with lysosomal storage, polyglucosan accumulation or abnormal glycogen accumulation. In some embodiments, the agent is for use in prevention or treatment of an autophagy-misregulation associated disease.

In some embodiments, the disease or the disorder is selected from the group consisting of: glycogen storage disease (GSD), adult polyglucosan body disease (APBD), and Lafora disease, Gaucher disease, Fabry disease, Tay-Sachs disease, Mucopolysaccharidoses (MPS) diseases, aspartylglucosaminuria, GM1-gangliosidosis, Krabbe (globoid cell leukodystrophy or galactosylceramide lipidosis), Metachromatic, leukodystrophy, Sandhoff disease, mucolipidosis type II (I-cell disease), mucolipidosis type IIIA (pseudo-Hurler poly dystrophy), Niemann-Pick disease type C2 and C1, Danon disease, free sialic acid storage disorder, mucolipidosis type IV, and multiple sulfatase deficiency (MSD), metabolic disorders, obesity, type II diabetes and insulin resistance.

In some embodiments, the agent is selected from the group consisting of:

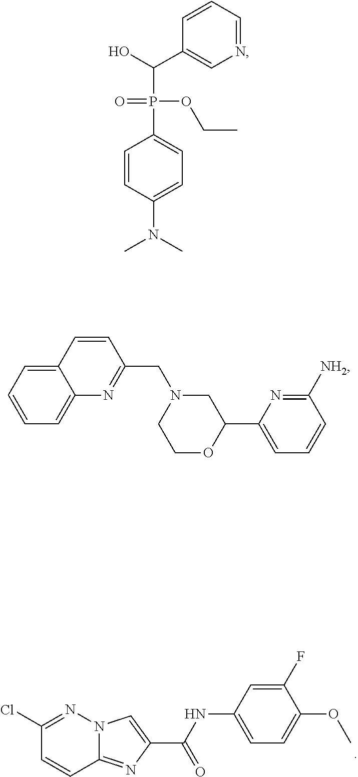 [SMILES: CCOP(=O)(c1ccc(N(C)C)cc1)C(O)c1cccnc1 ; Nc1cccc(C2CN(Cc3ccc4ccccc4n3)CCO2)n1 ; COc1ccc(NC(=O)c2cn3nc(Cl)ccc3n2)cc1F]

In another aspect of the invention, there is provided a pharmaceutical composition comprising the agent of the present invention and a pharmaceutically acceptable carrier.

In some embodiments, the pharmaceutical composition has a pH between 4 and 6.5, in solution.

In some embodiments, the pharmaceutical composition comprises between 100 nM and 5 mM of the agent.

In another aspect of the invention, there is provided a method for treating or preventing development of a disease or a disorder associated with lysosomal storage, polyglucosan accumulation or abnormal glycogen accumulation and an autophagy-misregulation associated disease in a subject in need thereof, comprising administering to the subject a therapeutically effective amount of the pharmaceutical composition of the present invention.

In another aspect of the invention, there is provided a method for determining suitability of a compound to prevent or treat a disease or a disorder associated with lysosomal storage, polyglucosan accumulation or abnormal glycogen accumulation and an autophagy-misregulation associated disease, the method comprising contacting the compound with a pocket domain within an N-terminal domain of a lysosomal-associated membrane protein 1 (LAMP-1; SEQ ID NO: 1), wherein binding of the compound to the pocket is indicative of the compound being effective in treat the disease or a disorder.

In some embodiments, the binding is to one or more of: SEQ ID NO: 2 (FSVNYD); and SEQ ID NO: 3 (NVTV).

In some embodiments, the binding is determined by inhibition of LAMP1:LAMP1 interaction.

In some embodiments, the binding is determined by inhibition of inter-LAMP1 interactions.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will prevail. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1M include the chemical structure of Compound 1 (1A), a graph of Kaplan-Meier survival curve based on 17 animals (n=17) treated twice a week with 250 mg/kg of Compound 1(144DG11) compared to 9 animals (n=9) treated with 5% DMSO vehicle (1B) (log-rank test p-value <0.000692), a graph of weight curve (in g) (1C), graph of average duration in movement in an open field (1D), and graph of extension reflex (the degree to which the hind paws open after holding the animal from the tail) as a function of time after treating wild type (n=8) mice with vehicle and Gbe^{ys/ys} mice with vehicle (n=8), or Compound 1 (n=9) as indicated (1E-1F); picture of Movement Heat Map (upper panel, average based on n=9, 9 month old females) showing quantification of Open Field performance experiments (1G), Lower panel shows visual tracking examples from single animals. wt., Untreated wild type animals as controls; tg treated, Gbe^{ys/ys} (transgenic) mice treated with Compound 1; tg, APBD mice treated with vehicle, graph of gait analysis (stride length) of n=9 nine months old female mice from each arm (1H); shown are average (+/−s.d.) stride lengths; graph of average duration in movement curve in an open field (1I), graph of weight (in g) curve (1J), and graph presenting extension reflex curve as a function of time after treating Gbe^{ys/ys} mice with vehicle or Compound 1 as indicated at 6 months of age (at onset) (1K). Pictures of 7-months old Gbe^{ys/ys} mice treated with vehicle (1L), or Compound 1 (1M) for 3 months prior to photography.

FIGS. 2A-2C include images and graphs of histopathological effects of Compound 1 and its pharmacokinetics: right panel present images of indicated tissues of sacrificed mice and stained for PG (arrows) with PAS following treatment with diastase; left panel, presents bar graphs quantifying PAS staining, based on analysis of 4 sections from each tissue in n=2 wild type, n=7 Gbe^{ys/ys} vehicle-treated, and n=9 Compound 1-treated mice (2A); bar graph quantifying total glycogen in the respective tissues (2B); graph of Compound 1pharmacokinetics (2C); 9 months old Gbe^{ys/ys} mice were SC-injected with 150 μL of Compound 1 at 250 mg/kg. Mice were sacrificed 30-, 60-, 90-, and 210-min post injection and the indicated tissues were removed, as well as 200 μL of serum drawn. Graph shows Compound 1 levels in the different tissues determined by LC-MS/MS. Shown are means and SEM of results obtained from n=3 mice at each time point. Repeated-measures 2-way ANOVA tests show that the pharmacokinetic profile of each tissue is significantly different from that of all other tissues (p<0.05). *, Significant difference (p<0.05) as determined by Student's t-tests.

FIGS. 3A-3I include a graph of the effect of Compound 1 on in vivo metabolism; mice were monitored by the Promethion High-Definition Behavioral Phenotyping System (Sable Instruments, Inc.) over a 24 hr period. Effective mass was calculated by power of 0.75. Data are mean±SEM from n=11 9 months old mice in the wt. control vehicle arm, n=6 9 months old mice in the GBE^{ys/ys} vehicle arm and n=7 9 months old mice in the Gbe^{ys/ys} 144DG11 (Compound 1)-treated arm. All injections were from the age of 4 months. Untreated GBE^{ys/ys} mice demonstrate lower respiratory quotient (in the light) (3A), total energy expenditure (TEE) (3B), and fat oxidation (3C) compared to wild type controls. Carbohydrate oxidation and ambulatory activity, not significantly affected by the diseased state, were increased by Compound 1 even beyond wt. control levels (3D-3E); Compound 1 has also reversed the decrease in meal size and water sip volume observed in Gbe^{ys/ys} mice as compared to wt. control (3F-3H). Blood metabolic panel based on n=5, 9.5 months old mice treated as indicated (3I). Blood glucose was increased, and blood triglycerides decreased in Gbe^{ys/ys} cells by Compound 1 (p<0.05, Student's t-tests). *p<0.05 v wt. controls, #p<0.05 v GBE^{ys/ys} vehicle treated mice;

FIGS. 4A-4D include a bar graph of PAS staining for total glycogen in skin fibroblasts from different APBD patients (4A), images of PAS staining for total glycogen in APBD87 fibroblasts glucose-starved for 48 h (left), or glucose starved and then replenished for the last 24 h to induce glycogen burden (right) (4B), image acquisition was done by Nikon Eclipse Ti2 microscope using a 40×PlanFluor objective and CY3 filter; graph of image-based multiparametric phenotyping of APBD fibroblasts under 48 h glucose-starvation, or starvation and glucose replenishment as in 4B (4C), level of significance p=0.01; and bar graph of glycolytic and mitochondrial ATP production determined by Agilent's Seahorse machine and ATP rate assay kit (4D). Healthy control (HC) and APBD patient fibroblasts were untreated or treated with 10 μM Compound 1 for 48 h (chronic), or on assay for 20 min (acute). Readings were normalized to cell number as determined by Crystal Violet staining. Shown are mean and SD values based on n=6 repeats.

FIGS. 5A-5E include images of experiments showing hetero-assembly forms around Compound 1 and not around endogenous molecules as shown by the liquid crystals formed in experiments 1-3 (5A); image of STRING network of targets at the interactome of Compound 1 (5B); cellular thermal shift assay (CETSA) of different targets of the Compound 1 hetero-assembly (5C); surface plasmon resonance sensograms of binding of Compound 1 to LAMP1 (5D); sensogram experiments consisting of association and dissociation at the indicated concentration ranges and pH values were then conducted. Results show that dose-responsive association of LAMP1 to Compound 1 started at pH 6, was partial at pH 5 and was clearly demonstrated at the lysosomal pH 4.5-5; images of three binding modes of Compound 1 according to LAMP 1 grids that were predicted by SiteMap, fPocket and FtSite (5E).

FIGS. 6A-6E include bar graphs of autophagic flux, determined by the extent of lysosomal inhibitors-dependent increase in the ratio of lipidated to non-lipidated LC3 (LC3II/LC3I) (6A); representative TEM images of liver tissue from 9.5 month old Gbe^{ys/ys} mice treated with Compound 11, or 5% DMSO vehicle (6B), G: Glycogen (alpha particles) and polyglucosan (structures with variable electron densities), L: Lysosomes, M: Mitochondria; right panel: lysosomal glycogen stain was quantified by ImageJ “count particle” tool; micrograph of LAMP1 knocked down and control APBD primary skin fibroblasts treated or not with Compound 1 and lysosomal inhibitors (LI) and quantification of 3 experiments and results of Student's t-tests. *, p<0.1; **, p<0.05; ***, p<0.01 (6C); graphs of lysosomal pH changes determined in APBD primary fibroblasts transduced with lentiviruses encoding for GFP or GFP-shLAMP1 and treated or not with Compound 1 for 24 h and confocal microscope images of cells treated with Lysosensor and stained for PAS (6D); and bar graph of ATP production rate assay in LAMP1-KD and GFP (Control) cells treated for 24 h (chronic) or on assay (acute) with Compound 1 (6E).

FIGS. 7A-7F include graphs of IBP parameters in HC and APBD fibroblasts and variable importance plot as an output of the random forest classification performed on the different variables (cell features) indicated on the x-axis. The random forest analysis has demonstrated APBD and HC cell populations to be separated at a confidence level of 93% (7A); graphs of multi-parametric cell-phenotypic characterization of n=5 HC and n=5 APBD patient skin fibroblasts: the extent of deviation from HC of the cell features shown, ordered by the amount of deviation (-log(P value)). Features where the value is above the dashed line (frame) demonstrate deviation from HC with a p value <0.01. The different comparisons analyzed (comp A=Compound 1) are shown (7B); bar graphs of lysosomal parameters affected by Compound 1 in APBD and HC cells as analyzed by IBP (7C); Volcano plots of the proteins affected by APBD and Compound 1 under starvation and glycogen burden conditions (7D); Venn diagrams of proteins down-modulated by APBD and up modulated by Compound 1 and vice versa under starvation (48) and glycogen burden (48+24) conditions (7E); and gene ontology of proteins up-modulated (left) and down-modulated (right) by Compound 1 (7F).

FIG. 8 includes in silico ADMET (Absorption, Distribution, Metabolism, and Excretion Toxicity)-compatible polyglucosan lowering compounds; analysis of three different ADMET algorithms;

FIG. 9 includes images of the result of an ADMET-incompatible compound (88095528 in FIG. 8) causing wounds in Gbe^{ys/ys} mice;

FIG. 10 includes a graph of the body weights of wild type C57B16J mice treated with Compound 1 for 3 months. Mice were injected twice a week with 150 μL of Compound 1 at 250 mg/kg in 5% DMSO, or an equal volume of 5% DMSO (V, vehicle) control. Injections were intravenous for the first month and then subcutaneously for the following 2 months;

FIG. 11 includes images of brain, liver, skeletal muscle, and heart tissue slices of wild type C57B16J mice treated for 3 months with Compound 1. The slices were stained by H&E staining in order to visualize lesions. No lesions were apparent in either treatment. Scale bars, 500 μm (brain), 100 μm (liver), 200 82 m (muscle), 100 μm (heart).

FIGS. 12A-12B include a micrograph of the glycosylation status of LAMP1 and RNase B tested by 15% SDS-PAGE mobility shift gel stained with QC colloidal Coomassie stain (#1610803, Bio-Rad) after short (24 h) or long (72 h) dialysis (12A); and a sensorgram showing the absence of interaction between deglycosylated LAMP1-Nter protein (degLAMP1-Nt) and Compound 11 (12B).

FIGS. 13A-13B include an image of Compound 1 predicted binding site in LAMP1′ s N-terminal domain and LAMP1 N-terminus:LAMP1 N-terminus protein:protein docking computations (13A), and a schematic representation of the lysosomal membrane (LM), LAMP1, LAMP2 and the potential inhibitor Compound 1 (13B).

FIGS. 14A-14B include images of the heteroassemblies (circles) obtained by NPOT® on APBD-patient fibroblasts (14A), or HC fibroblasts (14B) in the presence of compounds 1 and OKMW-XXC (negative control) at 10 -6 M. Each experiment was done in triplicate. Technical negative controls were obtained without the addition of any compound. Each picture represents a well of a 96-well plate.

FIG. 15 includes a micrograph and a vertical bar graph showing autophagic flux in PD patient-derived skin fibroblasts, serum starved and treated (or not) with 50 μM 144DG11 (indicated as comp. A).

FIG. 16 includes fluorescent micrographs and a vertical bar graph showing that treatment of PD primary fibroblasts with 144DG11 (50 μM, 24 h) significantly lowered PAS staining (magenta) indicating reduction of glycogen. Yellow, Calcein used for cell segmentation, Blue, DAPI nuclear stain. Middle panel shows quantification of segmented autophagic flux in PD patient-derived skin fibroblasts, serum starved and treated (or not) with 50 μM 144DG11 (indicated as comp. A).

FIG. 17 includes a vertical bar graph showing glycolytic (1) and mitochondrial (2) ATP production determined by Agilent's Seahorse machine and ATP rate assay kit. HC and PD patient fibroblasts were serum/glucose-starved for 48 h and then full medium was replenished for 24 h without (untreated), or with (chronic) 50 μM 144DG11. Acute, 50 μM 144DG11 was added on assay for 20 min after 24 h of serum/glucose replenishment. Readings were normalized to cell number as determined by Crystal Violet staining. Shown are mean and SD values based on n=6 repeats. In acutely 144DG11-treated PD fibroblasts, glycolytic and total ATP production was increased, as compared to untreated PD cells (p<0.002, One Way ANOVA with Sidak's post-hoc correction for multiple comparisons).

FIG. 18 includes a vertical bar graph showing blood metabolic panel based on n=5-6 6-months wildtype or Agl^{-/-} mice treated as indicated for 3 months. Blood triglycerides were decreased by 144DG11, suggesting correction of hyperlipidemia. *p<0.049, ** p<0.004 (t-tests).

FIGS. 19A-19B include fluorescent micrographs showing microglia cells which were isolated from the brains of AD modeling 5XFAD mice by CD11b magnetic beads. Microglia were then incubated for 24 h with (treated), or without (untreated) 50 μM 144DG11, fixed and stained for the autophagic substrates LC3 (19A) and p62 (19B) and for glycogen by PAS, all as indicated. Reduction in the levels of both LC3 and p62 indicate induction of autophagy which degrades these substrates.

FIGS. 20A-20B include fluorescent micrographs showing primary non-small cell-lung cancer. Cells were treated and stained for the autophagic substrates LC3 (20A) and p62 (20B) as in 19A-19B.

FIG. 21 includes vertical bar graphs showing skin fibroblasts derived from Gsd1a patients were treated with solvent or 50 μM Compound A for 24 h and analyzed for NAD+/NADH ratio by the Promega kit (left panel) and for Sirt1 (middle panel) and p62 (right panel) expression by western immunoblotting.

### DETAILED DESCRIPTION OF THE INVENTION

The present invention is directed to a pharmaceutical composition for use in prevention or treatment of a disease or a disorder associated with lysosomal storage.

The present invention further is directed to a pharmaceutical composition for use in prevention or treatment of a disease or a disorder associated with polyglucosan accumulation or abnormal glycogen accumulation.

The present invention further is directed to a pharmaceutical composition for use in prevention or treatment of a disease or a disorder associated with abnormal protein accumulation.

The present invention further is directed to a pharmaceutical composition for use in prevention or treatment of autophagy-misregulation associated diseases. The present invention further is directed to a pharmaceutical composition for use in prevention or treatment of a disease or a disorder associated with reduction in autophagy.

The present invention is also directed to an agent that binds a region of an N-terminal domain of a lysosomal-associated membrane protein 1 (LAMP-1).

The present invention is also directed to a method for treating or preventing development of a disease or a disorder associated with lysosomal storage, polyglucosan accumulation or abnormal glycogen accumulation in a subject in need thereof.

According to some embodiments, the present invention provides a compound, pharmaceutically acceptable salt, isomer or tautomer thereof, for use in prevention or treatment of a disease or a disorder selected from a lysosomal storage associated disease and an autophagy-misregulation associated disease, wherein the compound is represented by Formula I:

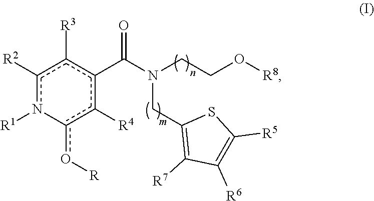

wherein:

[symbol] represents a single or a double bond; n and m each independently represents an integer in a range from 1 to 3; R and R¹ each independently represents hydrogen, or is absent; and R³, R⁴, R⁵, R⁶, R⁷ and R⁸ each independently represents hydrogen, or is selected from the group comprising alkyl, cycloalkyl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, substituted or non-substituted.

In some embodiments, either R or R¹ represents hydrogen. In some embodiments, R is hydrogen and R¹ is absent. In some embodiments, R¹ is hydrogen and R is absent.

In some embodiments, n and m is 1.

In some embodiments, R², R⁷ and R⁸ represent a methyl. In some embodiments, the compound is selected from:

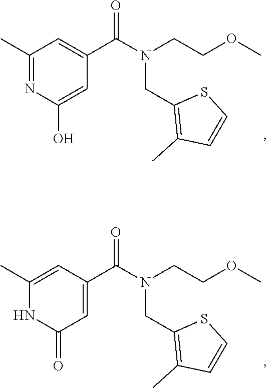

or both.

According to some embodiments, the present invention provides a pharmaceutical composition for use in prevention or treatment of a disease or a disorder selected from a lysosomal storage associated disease and an autophagy-misregulation associated disease, the pharmaceutical composition comprising a compound, pharmaceutically acceptable salt, isomer or tautomer thereof, wherein the compound is represented by Formula I, as described hereinabove.

According to some embodiments, the present invention provides a pharmaceutical composition for use in prevention or treatment of a disease selected from a disorder associated with lysosomal storage, obesity, type II diabetes and insulin resistance, the pharmaceutical composition comprising a compound, pharmaceutically acceptable salt, isomer or tautomer thereof, wherein the compound is represented by Formula I, as described hereinabove.

In some embodiments, a disease or a disorder associated with lysosomal storage refers to a disease or a disorder associated with the incapacity of lysosomal enzymes to break down accumulated substrates, swollen lysosomes, burst of lysosomes, compromised lysosomal signal transduction, or any combination thereof.

In some embodiments, the pharmaceutical composition is for use in prevention or treatment of a disease or a disorder associated with the incapacity of lysosomal enzymes to break down accumulated substrates. In some embodiments, the pharmaceutical composition is for use in prevention or treatment of a disease or a disorder associated with swollen lysosomes. In some embodiments, the pharmaceutical composition is for use in prevention or treatment of a disease or a disorder associated with burst of lysosomes, causing the spilling of toxic content into cytosol.

In some embodiments, the disease or the disorder associated with lysosomal storage is selected from the group consisting of: Gaucher disease, Fabry disease, Tay-Sachs disease, Mucopolysaccharidoses (MPS) diseases, aspartylglucosaminuria, GM1-gangliosidosis, Krabbe (globoid cell leukodystrophy or galactosylceramide lipidosis), Metachromatic, leukodystrophy, Sandhoff disease, mucolipidosis type II (I-cell disease), mucolipidosis type IIIA (pseudo-Hurler poly dystrophy), Niemann-Pick disease type C2 and Cl, Danon disease, free sialic acid storage disorder, mucolipidosis type IV, and multiple sulfatase deficiency (MSD), and metabolic disorders.

The terms “lysosomal storage”, “lysosomal storage diseases” and “lysosomal storage disorders” (LSDs) are used interchangeably herein to refer to a group of inherited diseases characterized by lysosomal dysfunction and neurodegeneration. These disorders are typically due to single gene defects: deficiency of specific enzymes that are normally required for the breakdown of glycosaminoglycans (GAGs), make the cell unable to excrete the carbohydrate residues, which thus accumulate in the lysosomes of the cell. This accumulation disrupts the cell's normal functioning and gives rise to the clinical manifestations of LSDs. Non-limiting examples of diseases or the disorders associated with lysosomal storage include Sphingolipidoses, Ceramidase (e.g., Farber disease, Krabbe disease), Galactosialidosis, gangliosidoses including Alpha-galactosidases (e.g., Fabry disease (alpha-galactosidase A), Schindler disease (alpha-galactosidase B)), Beta-galactosidase (e.g., GM1 gangliosidosis, GM2 gangliosidosis, Sandhoff disease, Tay-Sachs disease), Glucocerebrosidoses (e.g., Gaucher disease (Type I, Type II, Type III), Sphingomyelinase (e.g., Lysosomal acid lipase deficiency, Niemann-Pick disease), Sulfatidosis (e.g., Metachromatic leukodystrophy. Multiple sulfatase deficiency), Mucopolysaccharidoses (e.g., Type I (MPS I (Hurler syndrome, 1VIPS I S Scheie syndrome, MPS I H-S Hurler-Scheie syndrome), Type II (Hunter syndrome), Type III (Sanfilippo syndrome), Type IV (Morquio), Type VI (Maroteaux-Lamy syndrome), Type VII (Sly syndrome), Type IX (hyaluronidase deficiency)), mucolipidoses (e.g., Type I (sialidosis), Type II (I-cell disease), Type III (pseudo-Hurler polydystrophy/phosphotransferase deficiency), Type IV (mucolipidin 1 deficiency)), lipidoses (e.g., Niemann-Pick disease), Neuronal ceroid lipofuscinoses (e.g., Type 1 Santavuori-Haltia disease/ infantile NCL (CLN1 PPT1)), Type 2 Jansky-Bielschowsky disease/late infantile NCL (CLN2/LINCL TPP1), Type 3 Batten-Spielmeyer-Vogt disease/juvenile NCL (CLN3), Type 4 Kufs disease/adult NCL (CLN4), Type 5 Finnish Variant/late infantile (CLN5), Type 6 Late infantile variant (CLN6), Type 7 CLN7, Type 8 Northern epilepsy (CLN8), Type 8 Turkish late infantile (CLN8), Type 9 German/Serbian late infantile, Type 10 Congenital cathepsin D deficiency (CTSD)), Wolman disease, Oligosaccharidoses (e.g., Alpha-mannosidosis, Beta- mannosidosis, Aspartylglucosaminuria, Fucosidosis), lysosomal transport diseases (e.g., Cystinosis, Pycnodysostosis, Salla disease/sialic acid storage disease, Infantile free sialic acid storage disease), Type II Pompe disease, Type lib Danon disease), Cholesteryl ester storage disease, and the like.

In some embodiments, use of a compound represented by Formula I, pharmaceutically acceptable salt, isomer or tautomer thereof, in prevention or treatment of disease or the disorder associated with lysosomal storage does not include, or excludes, glycogen storage disease (GSD) or a condition associated therewith. In some embodiments, use of a compound represented by Formula I, pharmaceutically acceptable salt, isomer or tautomer thereof, in prevention or treatment of disease or the disorder associated with lysosomal storage does not include, or excludes, GSD type IV, GSD type VII, APDB, or any combination thereof.

In some embodiments, use of a compound represented by Formula I, pharmaceutically acceptable salt, isomer or tautomer thereof, in prevention or treatment of disease or the disorder associated with lysosomal storage does not include, or excludes, glycogen storage disease (GSD) associated neurodegenerative disease.

According to some embodiments, the present invention provides a method for treating or preventing development of a disease or a disorder associated with lysosomal storage in a subject in need thereof, comprising administering to the subject a therapeutically effective amount of the pharmaceutical composition described hereinabove.

In some embodiments, therapeutically effective amount is an amount effective to slow the progression, stop, or reverse protein accumulation/aggregation associated with the lysosomal storage disease or disorder. In some embodiments, therapeutically effective amount is an amount effective to slow the progression, stop, or reverse polyglucosan accumulation or abnormal glycogen accumulation. In some embodiments, therapeutically effective amount is an amount effective to increase autophagic activity.

In some embodiments, therapeutically effective amount is an amount effective to ameliorate one or more symptoms of the pathology associated with the lysosomal storage disease and/or to reduce neurodegeneration and/or neuro-inflammation associated with the lysosomal storage disease.

In another aspect, the present invention provides a method for treating or preventing development of a disease or a disorder associated with reduced or mis-regulated autophagic activity.

In some embodiments, the autophagy-misregulation associated disease is a disease caused by misfolded protein aggregates. In another embodiment of this aspect, the disease caused by misfolded protein aggregates is selected from the group including: Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease.

The term “autophagy-misregulation associated disease” also includes any disease or disorder including but not limited to cancer, cardiovascular, neurodegenerative, metabolic, pulmonary, renal, infectious, musculoskeletal, and ocular disorders, wherein the induction of autophagy would contribute to delaying the onset, slowing, stopping, or reversing the progression of one or more of symptoms associated with the disease or disorder.

The term “autophagy-misregulation associated disease” also includes cancer, e.g., any cancer wherein the induction of autophagy would inhibit cell growth and division, reduce mutagenesis, remove mitochondria and other organelles damaged by reactive oxygen species or kill developing tumor cells. The term “autophagy-misregulation associated disease” also includes a psychiatric disease or disorder, e.g., any psychiatric disease or disorder wherein the induction of autophagy would contribute to delaying the onset, slowing, stopping, or reversing the progression of one or more of symptoms associated with the psychiatric disease or disorder. In one embodiment, the psychiatric disease or disorder is selected from schizophrenia and a bipolar disorder.

In one aspect, the present invention discloses a method of inducing autophagy in a cell, the method comprising contacting the cell with the pharmaceutical composition of the invention in an amount effective to induce autophagy in the cell.

In one embodiment, the cell is present in a subject. In another embodiment, the cell is present in an in vitro cell culture. Non-limiting examples of the cell are neural cells, glial cells, such as astrocytes, oligodendrocytes, ependymal cells, Schwann cells, lymphatic cells, epithelial cells, endothelial cells, lymphocytes, cancer cells, and haematopoietic cells.

The term “autophagy” refers to the catabolic process involving the degradation of a cell's own components; such as, long lived proteins, protein aggregates, cellular organelles, cell membranes, organelle membranes, and other cellular components. The mechanism of autophagy may include: (i) the formation of a membrane around a targeted region of the cell, separating the contents from the rest of the cytoplasm, (ii) the fusion of the resultant vesicle with a lysosome and the subsequent degradation of the vesicle contents.

In some embodiments, there is provided a method for reducing neurodegeneration, reducing neuro-inflammation, slowing the progression, or reducing memory-deficit, reducing abnormal lysosome size, re-activating autophagic flux, or any combination thereof, the method comprising administering to the subject a therapeutically effective amount of the pharmaceutical composition described hereinabove.

In some embodiments, the method comprises re-activating autophagic flux is in a subject afflicted with a disease or a disorder wherein autophagy is perturbed. In some embodiments, the method comprises re-activating autophagic flux is in a subject afflicted with LDS, as disclosed herein. In some embodiments, the method comprises re-activating autophagic flux is in a subject afflicted with Pompe disease. In some embodiments, the cancer is a cancer associated with reduced autophagic activity.

In some embodiments, there is provided a method for ameliorating one or more symptoms selected from the group consisting of leukodystrophy, scoliosis, hepatosplenomegaly, psychomotor regression, and ichthyosis, and/or delaying the onset, slowing, stopping, or reversing the progression of one or more of these symptoms.

In some embodiments, the subject is identified as having the lysosomal storage disease by the presence of a genetic marker for the lysosomal storage disease.

In some embodiments, administering is within 1 month of birth, 2 moths of birth, 3 months of birth, 6 months of birth, 1 year of birth, or within 3 years of birth, including any value therebetween. Each possibility represents a separate embodiment of the invention.

In some embodiments, the compounds and pharmaceutical compositions as described hereinabove are capable of inhibiting and/or modulating aggregation of one or more proteins, and/or promoting disaggregation of protein fibrils or other protein aggregates, or both. In some embodiments the compounds and pharmaceutical compositions as described hereinabove are capable of inhibiting and/or modulating aggregation of one or more amyloidogenic proteins (e.g., one or more of a-synuclein, Ab, tau, and the like), and/or promoting disaggregation of amyloid protein fibrils or other amyloid protein aggregates, or both.

### Lysosomal Membrane Protein 1 (LAMP1) Targeting Agents

According to some embodiments, the present invention provides an agent that binds a region of an N-terminal domain of a lysosomal-associated membrane protein 1 (LAMP-1; SEQ ID NO: 1;

FSVNYDTKSGPKNMTFDLPSDATVVLNRSSCGKENTSDPSLVIAFGRGH
TLTLNFTRNATRYSV).

As used herein, LAMP1 relates to Lysosome-associated membrane glycoprotein 1 having UniProt Accession no. P11279. In some embodiments, the LAMP1 has the amino acid sequence as set forth in SEQ ID NO: 4

(MAAPGSARRPLLLLLLLLLLGLMHCASAAMFMVKNGNGTACIMANFSA
AFSVNYDTKSGPKNMTFDLPSDATVVLNRSSCGKENTSDPSLVIAFGRG
HTLTLNFTRNATRYSVQLMSFVYNLSDTHLFPNASSKEIKTVESITDIR
ADIDKKYRCVSGTQVHMNNVTVTLHDATIQAYLSNSSFSRGETRCEQDR
PSPTTAPPAPPSPSPSPVPKSPSVDKYNVSGINGTCLLASMGLQLNLTY
ERKDNTTVTRLLNINPNKTSASGSCGAHLVTLELHSEGTTVLLFQFGMN
ASSSRFFLQGIQLNTILPDARDPAFKAANGSLRALQATVGNSYKCNAEE
HVRVTKAFSVNIFKVWVQAFKVEGGQFGSVEECLLDENSMLIPIAVGGA
LAGLVLIVLIAYLVGRKRSHAGYQTI).

In some embodiments, the agent binds at least one region of LAMP1 selected from any one of: SEQ ID NO: 2 (FSVNYD); and SEQ ID NO: 3 (NVTV) or a homolog thereof.

In some embodiments, the agent binds an amino acid residue selected from residues F50-D55, N62, L67, F118, Y120-L122, T125, L127-5133, N164-V166 of LAMP-1 (i.e., of SEQ ID NO: 4). In some embodiments, the agent binds a combination of amino acid residue selected from residues F50-D55, N62, L67, F118, Y120-L122, T125, L127-S133, N164-V166 of LAMP-1 (i.e., of SEQ ID NO: 4).

As used herein, a homolog of SEQ ID NO: 2 (FSVNYD); and SEQ ID NO: 3 (NVTV) refers to at least one mutation (e.g., substitution) for which the agent can still bind a pocket region of an N-terminal domain of a LAMP-1 (SEQ ID NO: 1) and provide the desired biological or pharmaceutical effect (e.g., hinder or inhibit a LAMP1:LAMP1 interaction or inhibits inter-LAMP1 interactions).

In some embodiments, a region of an N-terminal domain of a LAMP-1 is a pocket.

Non-limiting examples for identifying the pocket include the following algorithms utilized by SiteMap, FtSite, or fPocket. In some embodiments, the pocket is identified using SiteMap, FtSite, or fPocket program.

As used herein, the term “pocket” refers to a cavity, indentation, or depression in the surface of a protein molecule that is created as a result of the folding of the peptide chain into the 3-dimensional structure that makes the protein functional. A pocket can readily be recognized by inspection of the protein structure and/or by using commercially available modeling software's.

The term “agent” as used herein refers to any small organic molecule capable of entering and/or binding to a protein pocket as described hereinabove.

As used herein, the term “small organic molecule” refers to a molecule of a size comparable to those organic molecules generally used in pharmaceuticals. The term excludes natural biological macromolecules (e.g., proteins, nucleic acids, etc.). In some embodiments, organic molecules have a size up to 5,000 Da, up to 2,000 Da, or up to 1,000 Da, including any value therebetween. Each possibility represents a separate embodiment of the invention.

In some embodiments, the agent is not a compound represented by Formula I.

In some embodiments, the agent is selected from the group consisting of:

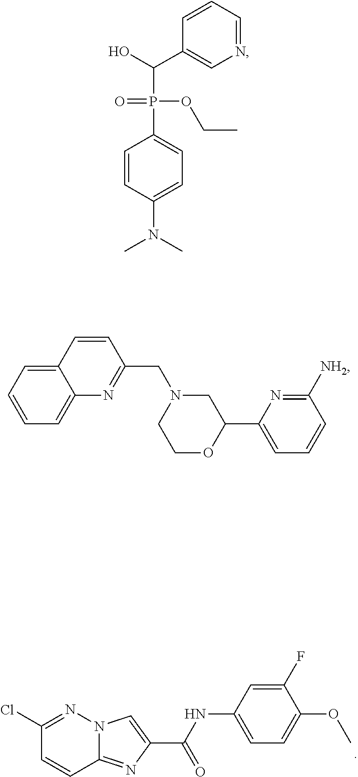 [SMILES: CCOP(=O)(c1ccc(N(C)C)cc1)C(O)c1cccnc1 ; Nc1cccc(C2CN(Cc3ccc4ccccc4n3)CCO2)n1 ; COc1ccc(NC(=O)c2cn3nc(Cl)ccc3n2)cc1F]

In some embodiments, the binding is specific binding.

The terms “specific binding” or “preferential binding” refer to that binding which occurs between two paired species (such as enzyme/substrate, receptor/agonist, antibody/antigen, and lectin/carbohydrate) which may be mediated by covalent and/or non-covalent interactions. When the interaction of the two species typically produces a non-covalently bound complex, the binding which occurs is typically electrostatic, and/or hydrogen-bonding, and/or the result of lipophilic interactions. Accordingly, “specific binding” occurs between pairs of species where there is interaction between the two that produces a bound complex. In particular, the specific binding is characterized by the preferential binding of one member of a pair to a particular species as compared to the binding of that member of the pair to other species within the family of compounds to which that species belongs. Thus, for example, an agent may show an affinity for a particular pocket on a LAMP-1 molecule (i.e., the pocket defined herein) that is at least two-fold preferably, at least 10-fold, at least 100-fold, at least 1,000-fold, or at least 10,000-fold greater than its affinity for a different pocket on the same or related proteins, including any value therebetween. Each possibility represents a separate embodiment of the invention.

In some embodiments, the agent inhibits a LAMPl:LAMP1 interaction. In some embodiments, the agent inhibits inter-LAMP1 interactions.

In some embodiments, the agent is for use in prevention or treatment of a disease or a disorder selected from a disease or a disorder associated with lysosomal storage, a disease or a disorder associated with polyglucosan accumulation or abnormal glycogen accumulation, and abnormal protein accumulation, and an autophagy-misregulation associated disease.

In some embodiments, the agent is for use in prevention or treatment of a disease or a disorder associated with the incapacity of lysosomal enzymes to break down accumulated substrates. In some embodiments, the agent is for use in prevention or treatment of a disease or a disorder associated with swollen lysosomes. In some embodiments, the agent is for use in prevention or treatment of a disease or a disorder associated with burst of lysosomes, causing the spilling of toxic content into cytosol.

In some embodiments, the disease or the disorder is selected from the group consisting of: glycogen storage disease (GSD), adult polyglucosan body disease (APBD), and Lafora disease, Gaucher disease, Fabry disease, Tay-Sachs disease, Mucopolysaccharidoses (MPS) diseases, aspartylglucosaminuria, GM1-gangliosidosis, Krabbe (globoid cell leukodystrophy or galactosylceramide lipidosis), Metachromatic, leukodystrophy, Sandhoff disease, mucolipidosis type II (I-cell disease), mucolipidosis type IIIA (pseudo-Hurler poly dystrophy), Niemann-Pick disease type C2 and C1, Danon disease, free sialic acid storage disorder, mucolipidosis type IV, and multiple sulfatase deficiency (MSD), metabolic disorders, obesity, and insulin resistance.

In some the disease or disorder is glycogen storage disorder (GSD). In some embodiments, the GSD is associated with glycogen-branching enzyme deficiencies. In some embodiments, the GSD is selected from types I-XV GSD. In some embodiments, the GSD is GSD type 0. In some embodiments, the GSD is GSD type 1. In some embodiments, the GSD is GSD type 2. In some embodiments, the GSD is GSD type 3. In some embodiments, the GSD is GSD type 4. In some embodiments, the GSD is GSD type 5. In some embodiments, the GSD is GSD type 6. In some embodiments, the GSD is GSD type 7. In some embodiments, the GSD is GSD type 9. In some embodiments, the GSD is GSD type 10. In some embodiments, the GSD is GSD type 11. In some embodiments, the GSD is GSD type 12. In some embodiments, the GSD is GSD type 13. In some embodiments, the GSD is GSD type 14 (also classed as Congenital disorder of glycosylation type 1 (CDG1T)). In some embodiments, the GSD is GSD type 15.

In some embodiments, the medical condition is one or more from, without being limited thereto, adult polyglucosan body disorder (APBD), Andersen disease, Forbes disease, and Danon disease.

In some embodiments, by GSD, or by “medical condition associated with “glycogen-branching enzyme deficiencies”, it is meant to refer to diseases or disorders characterized by deposition, accumulation or aggregation of polyglucosan bodies in muscle, nerve and/or various other tissues of the body. In some embodiments, the medical condition is characterized by dysfunction of the central and/or peripheral nervous systems of a subject.

Various methods for personalizing treatment, prevention, or reduction of the incidence or severity of GSD and other disorders related to the accumulation of polyglucosan bodies are encompassed in embodiments of the invention.

In some embodiments, the agent is used to treat neurodegenerative diseases. In some embodiments, the agent is used to treat inflammatory diseases. In some embodiments, the agent is used to treat GSD-associated cancer.

In some embodiments, the cancer is a cancer associated with reduced autophagic activity. In some embodiments, cancer comprises or is lung cancer. In some embodiments, lung cancer is or comprises non-small cell lung cancer (NSCLC).

In some embodiments, the agent is characterized by an activity that decreases polyglucosan body (PB) cellular content. In some embodiments, by “decreases PB cellular content”, it is meant to refer to shaping (e.g., reducing) the size of PB. In some embodiments, by “decreases PB cellular content”, it is meant to refer to degrading the PB. (e.g., by modulating glycogen branching enzyme, GBE).

In some embodiments, the agent is capable of modulating (e.g., inhibiting, or in some embodiment, increasing) an activity of at least one enzyme.

In some embodiments, the agent is capable of inhibiting one or more enzymes. Non-limiting examples of such enzyme is glycosyltransferase e.g., glycogen synthase (GS) and protein phosphatase-1 (PP1).

In some embodiments, the autophagy-misregulation associated disease is a disease caused by misfolded protein aggregates. In another embodiment of this aspect, the disease caused by misfolded protein aggregates is selected from the group including: Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease. The term “autophagy-misregulation associated disease” also includes cancer, e.g., any cancer wherein the induction of autophagy would inhibit cell growth and division, reduce mutagenesis, remove mitochondria and other organelles damaged by reactive oxygen species or kill developing tumor cells. The term “autophagy-misregulation associated disease” also includes a psychiatric disease or disorder, e.g., any psychiatric disease or disorder wherein the induction of autophagy would contribute to delaying the onset, slowing, stopping, or reversing the progression of one or more of symptoms associated with the psychiatric disease or disorder. In one embodiment, the psychiatric disease or disorder is selected from schizophrenia and a bipolar disorder.

The term “inhibitory” or any grammatical derivative thereof, as used herein in the context of enzymes refers to being capable of preventing, blocking, attenuating, or reducing the activity of an enzyme.

In some embodiments, by “reducing the activity”, it is meant to refer to an activity being reduced by at least 20%, at least 30%, at least 40%, at least 50%, at least 60%, at least 70%, at least 80%, or at least 90%, including any value and range therebetween, relative to comparable situation lacking the presence of the disclosed compound or a composition of matter containing same.

The disclosed agents, alone or in combination thereof or with any another therapeutically active agent, can be designed and utilized to exert a dual and possibly synergistic activity when in combination thereof or with any another therapeutically active agent.

According to some embodiments, the present invention provides a pharmaceutical composition comprising the agent described hereinabove.

In some embodiments, the pharmaceutical composition has a pH between 4 and 6.5, between 4.5 and 6.5, between 4 and 6, between 4 and 5.5, between 4 and 5, between 4.5 and 6, between 4.5 and 5.5, or between 4.5 and 5, in solution, including any range therebetween. Each possibility represents a separate embodiment of the invention.

In some embodiments, the agent shows specific binding to LAMP-1 at a pH between 4 and 6.5, between 4.5 and 6.5, between 4 and 6, between 4 and 5.5, between 4 and 5, between 4.5 and 6, between 4.5 and 5.5, or between 4.5 and 5, in solution, including any range therebetween. Each possibility represents a separate embodiment of the invention.

In some embodiments, the agent shows specific binding to LAMP-1 at a lysosomal pH between 4 and 6.5, between 4.5 and 6.5, between 4 and 6, between 4 and 5.5, between 4 and 5, between 4.5 and 6, between 4.5 and 5.5, or between 4.5 and 5, in solution, including any range therebetween. Each possibility represents a separate embodiment of the invention.

In some embodiments, the pharmaceutical composition comprises between 100 nM and 5 mM, between 150 nM and 5 mM, between 200 nM and 5 mM, between 500 nM and 5 mM, between 700 nM and 5 mM, between 900 nM and 5 mM, between 1 mM and 5 mM, between 2 mM and 5 mM, between 100 nM and 3 mM, between 150 nM and 3 mM, between 200 nM and 3 mM, between 500 nM and 3 mM, between 700 nM and 3 mM, between 900 nM and 3 mM, between 1 mM and 3 mM, between 2 mM and 3 mM, between 100 nM and 1 mM, between 150 nM and 1 mM, between 200 nM and 1 mM, between 500 nM and 1 mM, or between 700 nM and 1 mM, of the agent, including any range therebetween. Each possibility represents a separate embodiment of the invention.

According to some embodiments, the present invention provides a method for treating or preventing development of a disease or a disorder associated with lysosomal storage, polyglucosan accumulation or abnormal glycogen accumulation in a subject in need thereof, comprising administering to the subject a therapeutically effective amount of the pharmaceutical composition described hereinabove.

In some embodiments, the disease or the disorder associated with lysosomal storage is selected from the group consisting of: Gaucher disease, Fabry disease, Tay-Sachs disease, Mucopolysaccharidoses (MPS) diseases, aspartylglucosaminuria, GM1-gangliosidosis, Krabbe (globoid cell leukodystrophy or galactosylceramide lipidosis), Metachromatic, leukodystrophy, Sandhoff disease, mucolipidosis type II (I-cell disease), mucolipidosis type IIIA (pseudo-Hurler poly dystrophy), Niemann-Pick disease type C2 and C1, Danon disease, free sialic acid storage disorder, mucolipidosis type IV, and multiple sulfatase deficiency (MSD), metabolic disorders, obesity, and insulin resistance.

In some embodiments, the invention provides a method for treating or preventing development of forms of GSD, including, but not limited to, GSD-IV, -VI, IX, XI and cardiac glycogenosis due to AMP-activated protein kinase gamma subunit 2 deficiency. In some embodiments, the disclosed compounds may reduce pathogenic PB accumulation in the PB involving GSDs, GSD type IV (APBD and Andersen disease), GSD type VII (Tarui disease), and Lafora Disease (LD).

As used herein a “lysosomal membrane protein” refers to LAMP-1, LAMP-2, CD63/LAMP-3, DC-LAMP, or any lysosomal associated membrane protein, or homologs, orthologs, variants (e.g., allelic variants) and modified forms (e.g., comprising one or more mutations, either naturally occurring or engineered). In one aspect, a LAMP polypeptide is a mammalian lysosomal associated membrane protein, e.g., such as a human or mouse lysosomal associated membrane protein. More generally, a “lysosomal membrane protein” refers to any protein comprising a domain found in the membrane of an endosomal/lysosomal compartment or lysosome-related organelle and which further comprises a lumenal domain.

### Pharmaceutical Compositions Comprising the Disclosed Compounds and Agents

According to an aspect of embodiments of the invention there is provided a pharmaceutical composition comprising one or more compounds and/or agents as described herein and a pharmaceutically acceptable carrier.

According to an aspect of embodiments of the invention there is provided a pharmaceutical composition comprising therapeutically effective amount of one or more compounds and/or agents as described herein.

As used herein, the phrase “therapeutically effective amount” describes an amount of the compound being administered which will relieve to some extent one or more of the symptoms of the condition being treated.

The term “subject” (which is to be read to include “individual”, “animal”, “patient” or “mammal” where context permits) defines any subject, particularly a mammalian subject, for whom treatment is indicated. In some embodiments, the subject is a human.

The compounds described hereinabove may be administered or otherwise utilized either as is, or as a pharmaceutically acceptable salt, an enantiomer, a tautomer, a diastereomer, a protonated or non-protonated form, a solvate, a hydrate, or a prodrug thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound. The neutral forms of the compounds may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of the present invention.

The phrase “pharmaceutically acceptable salts” is meant to encompass salts of the active compounds which are prepared with relatively nontoxic acids or bases, depending on the particular substituents found on the compounds described herein.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compound as described herein to be converted into either base or acid addition salts.

In some embodiments, the neutral forms of the compounds described herein are regenerated by contacting the salt with a base or acid and isolating the parent compounds in a conventional manner. The parent form of the compounds differs from the various salt forms in certain physical properties, such as solubility in polar solvents, but otherwise the salts are equivalent to the parent form of the compound for the purposes of the present invention.

The term “prodrug” refers to an agent, which is converted into the active compound (the active parent drug) in vivo. Prodrugs are typically useful for facilitating the administration of the parent drug. The prodrug may also have improved solubility as compared with the parent drug in pharmaceutical compositions. Prodrugs are also often used to achieve a sustained release of the active compound in vivo.

In some embodiments, the compounds described herein possess asymmetric carbon atoms (optical centers) or double bonds; the racemates, diastereomers, tautomers, geometric isomers and individual isomers are encompassed within the scope of the present invention.

As used herein and in the art, the term “enantiomer” describes a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems.

In some embodiments, the compounds described herein can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

In some embodiments, the “pharmaceutical composition” refers to a preparation of one or more of the compounds described herein (as active ingredient), or physiologically acceptable salts or prodrugs thereof, with other chemical components including, but not limited to, physiologically suitable carriers, excipients, lubricants, buffering agents, antibacterial agents, bulking agents (e.g., mannitol), antioxidants (e.g., ascorbic acid or sodium bisulfate), anti-inflammatory agents, anti-viral agents, chemotherapeutic agents, anti-histamines and other.

In some embodiments, the purpose of a pharmaceutical composition is to facilitate administration of a compound to a subject. The term “active ingredient” refers to a compound, which is accountable for a biological effect.

The terms “physiologically acceptable carrier” and “pharmaceutically acceptable carrier”, which may be interchangeably used, refer to a carrier or a diluent that does not cause significant irritation to an organism and does not abrogate the biological activity and properties of the administered compound.

Herein the term “excipient” refers to an inert substance added to a pharmaceutical composition to further facilitate administration of a drug. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, PA, latest edition, which is incorporated herein by reference.

In some embodiments, pharmaceutical compositions for use in accordance with the present invention thus may be formulated in conventional manner using one or more pharmaceutically acceptable carriers comprising excipients and auxiliaries, which facilitate processing of the compounds into preparations which can be used pharmaceutically. Proper formulation is dependent upon the route of administration chosen. The dosage, as described and specified herein, may vary depending upon the dosage form employed and the route of administration utilized. The exact formulation, route of administration and dosage can be chosen by the individual physician in view of the patient's condition (see e.g., Fingl et al., 1975, in “The Pharmacological Basis of Therapeutics”, Ch. 1 p.1).

In some embodiments, the pharmaceutical composition may be formulated for administration in either one or more of routes depending on whether local or systemic treatment or administration is of choice, and on the area to be treated. As further described herein throughout, administration may be done orally, dentally, by inhalation, or parenterally, for example by intravenous drip or intraperitoneal, subcutaneous, intramuscular or intravenous injection, or topically (including ophtalmically, vaginally, rectally, intranasally).

Formulations for topical and/or dental administration may include but are not limited to lotions, ointments, gels, creams, suppositories, drops, liquids, sprays and powders. Conventional pharmaceutical carriers, aqueous, powder or oily bases, thickeners and the like may be necessary or desirable.

Compositions for oral administration may include powders or granules, suspensions, dental compositions, or solutions in water or non-aqueous media, sachets, pills, caplets, capsules or tablets. Thickeners, diluents, flavorings, dispersing aids, emulsifiers or binders may be desirable.

Formulations for parenteral administration may include, but are not limited to, sterile solutions which may also contain buffers, diluents and other suitable additives. Slow release compositions are envisaged for treatment.

The amount of a composition to be administered will, of course, be dependent on the subject being treated, the severity of the affliction, the manner of administration, the judgment of the prescribing physician, etc.

The pharmaceutical composition may further comprise additional pharmaceutically active or inactive agents such as, but not limited to, an antibacterial agent, an antioxidant, a buffering agent, a bulking agent, a surfactant, an anti-inflammatory agent, an anti-viral agent, a chemotherapeutic agent and anti-histamine.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be recognized that these embodiments are susceptible to various modifications and alternative forms well known to those of skill in the art.

### Screening Method

According to an aspect of some embodiments of the present invention, there is provided a method for determining suitability of a compound to prevent or treat a disease or a disorder associated with lysosomal storage, a disease or a disorder associated with polyglucosan accumulation or abnormal glycogen accumulation, and abnormal protein accumulation, and an autophagy-misregulation associated disease, the method comprising contacting the compound with a pocket domain within an N-terminal domain of a lysosomal-associated membrane protein 1 (LAMP-1; SEQ ID NO: 1), wherein binding of the compound to the pocket is indicative of the compound being effective in treating the disease or a disorder.

In some embodiments, the binding is to one or more of: SEQ ID NO: 2 (FSVNYD); and SEQ ID NO: 3 (NVTV).

In some embodiments, the binding is determined by inhibition of LAMPl:LAMP1 interaction.

In some embodiments, the binding is determined by inhibition of inter-LAMP1 interactions.

In some embodiments, the method comprises a step of computational screening of libraries of compounds.

In some embodiments, the method comprises detecting reduction of PB exerted by one or more selected compound (e.g., a small molecule).

It will be appreciated that by virtue of enabling computational screening of libraries of compounds having essentially any of various chemical, biological and/or physical characteristics, the method enables identification of a compound capable of displaying optimal in-vivo pharmacokinetics, optimally low immunogenicity, and optimal effectiveness relative to all prior art compounds capable of decreasing PB cellular content, for example, by correcting impaired enzymatic activity associated with glycogen storage disease e.g., glycogen synthase or, glycogen branching enzyme.

In some embodiments, the method comprises biochemically qualifying the capacity of the compound to decrease PB cellular content.

In some embodiments, the biochemically qualifying comprises subjecting cells to Periodic Acid-Schiff (PAS) staining to provide PAS-stained cells. In some embodiments, the method further comprises washing the sample to remove unreacted Schiff s reagents followed by detecting a signal (e.g., light fluorescing) derived from the PAS-stained sample at a defined wavelength.

Further embodiments of the disclosed method are provided in the Examples section below.

### Definitions

As used herein, the term “alkyl” describes an aliphatic hydrocarbon including straight chain and branched chain groups. Preferably, the alkyl group has 21 to 100 carbon atoms, and more preferably 21-50 carbon atoms. Whenever a numerical range; e.g., “21-100”, is stated herein, it implies that the group, in this case the alkyl group, may contain 21 carbon atoms, 22 carbon atoms, 23 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated.

The term “halide”, “halogen” or “halo” describes fluorine, chlorine, bromine or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” or “sulfonate” group describes an —S(═O)₂—R′ group, where Rx is as defined herein.

A “carbamyl” or “carbamate” group describes an —OC(═O)—NR′R″ group, where R′ is as defined herein and R″ is as defined for R′.

A “nitro” group refers to a —NO₂ group.

A “cyano” or “nitrile” group refers to a —C≡N group.

As used herein, the term “azide” refers to a —N₃ group.

The term “sulfonamide” refers to a —S(═O)₂—NR′R″ group, with R′ and R″ as defined herein.

The term “phosphonyl” or “phosphonate” describes an —O—P(═O)(OR′)₂ group, with R′ as defined hereinabove.

The term “phosphinyl” describes a —PR′R″ group, with R′ and R″ as defined hereinabove.

The term “alkaryl” describes an alkyl, as defined herein, which substituted by an aryl, as described herein. An exemplary alkaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion. Materials and Methods Study design

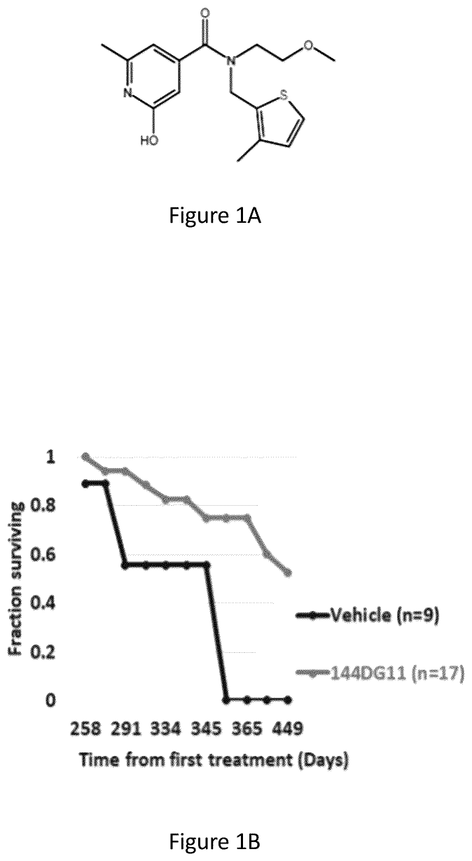

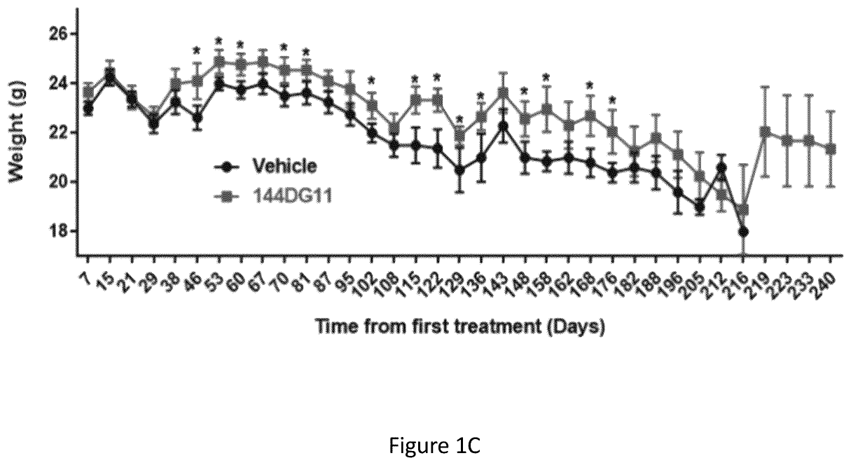

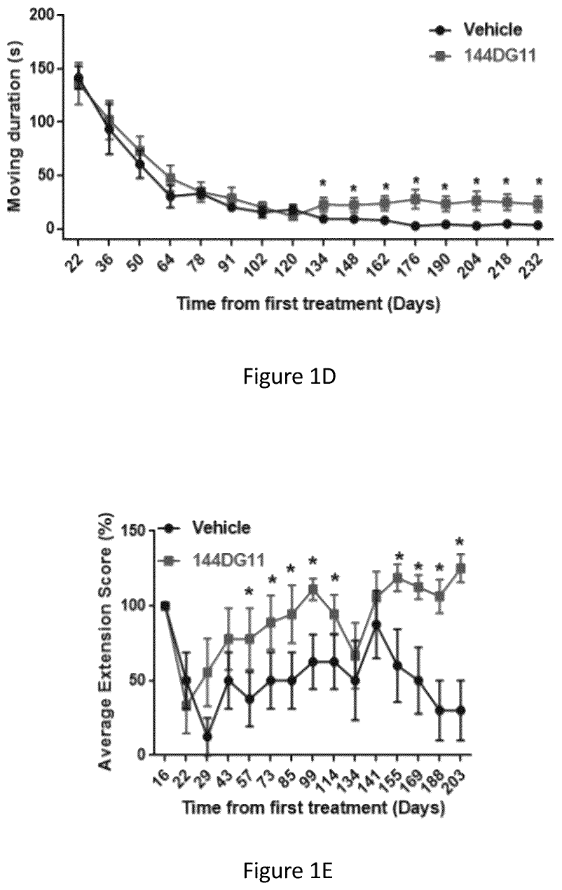

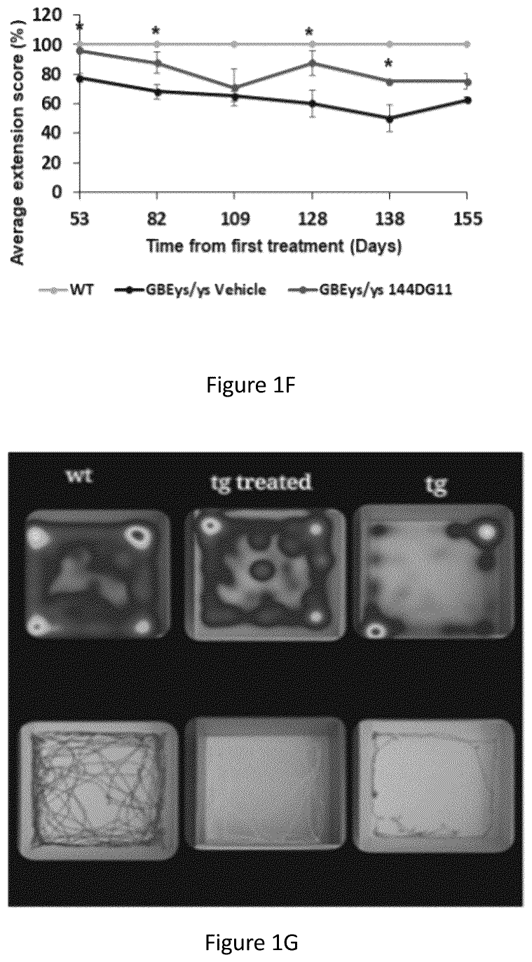

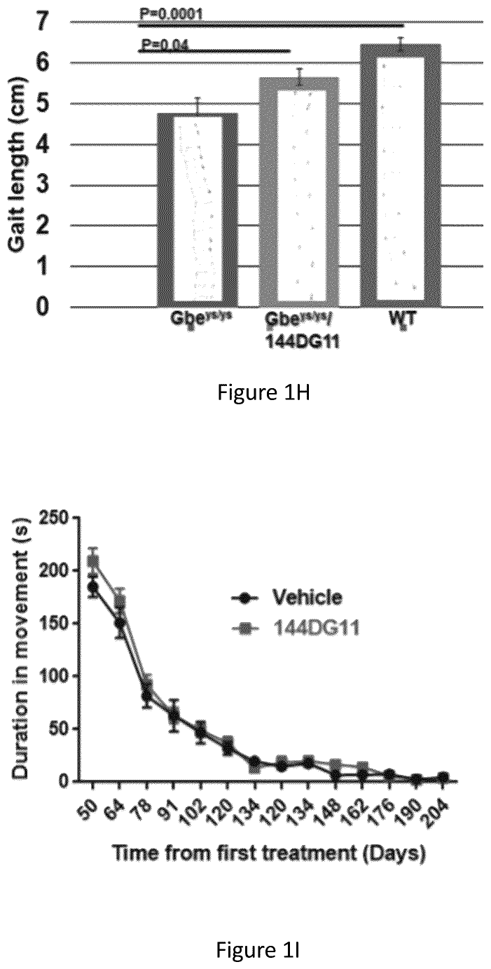

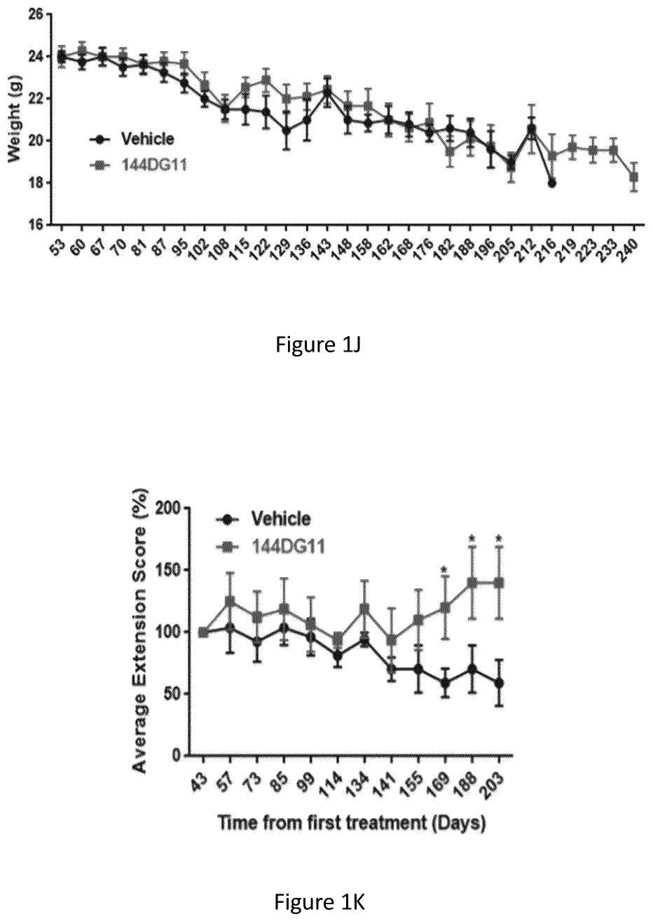

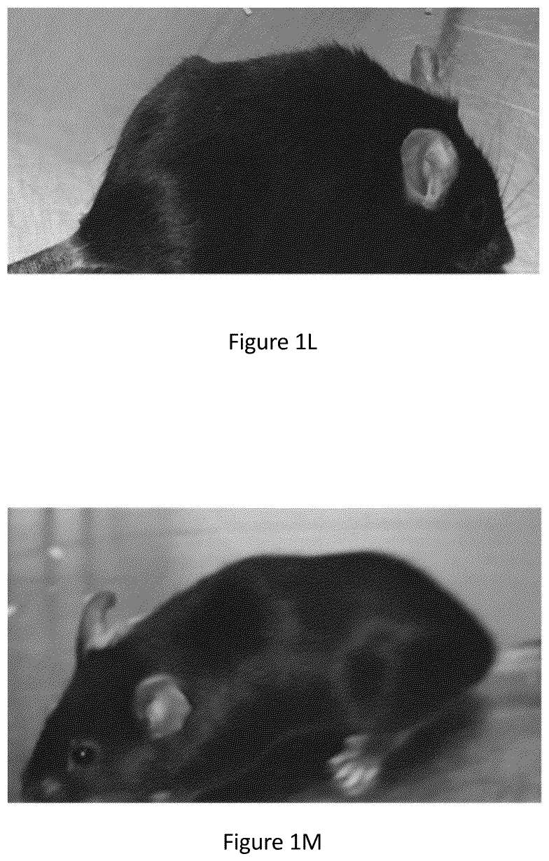

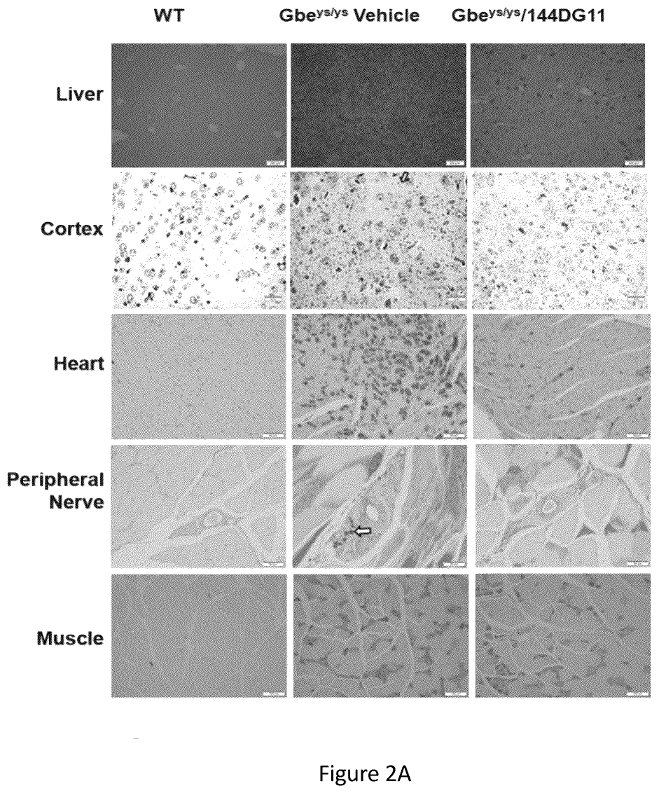

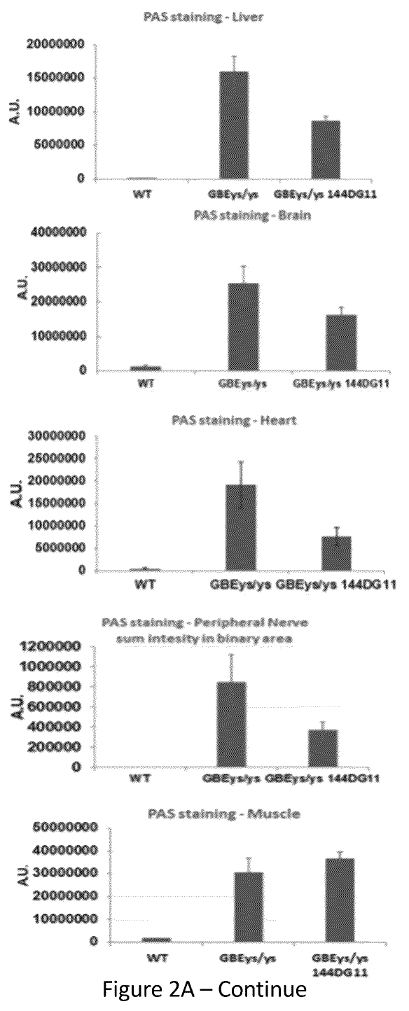

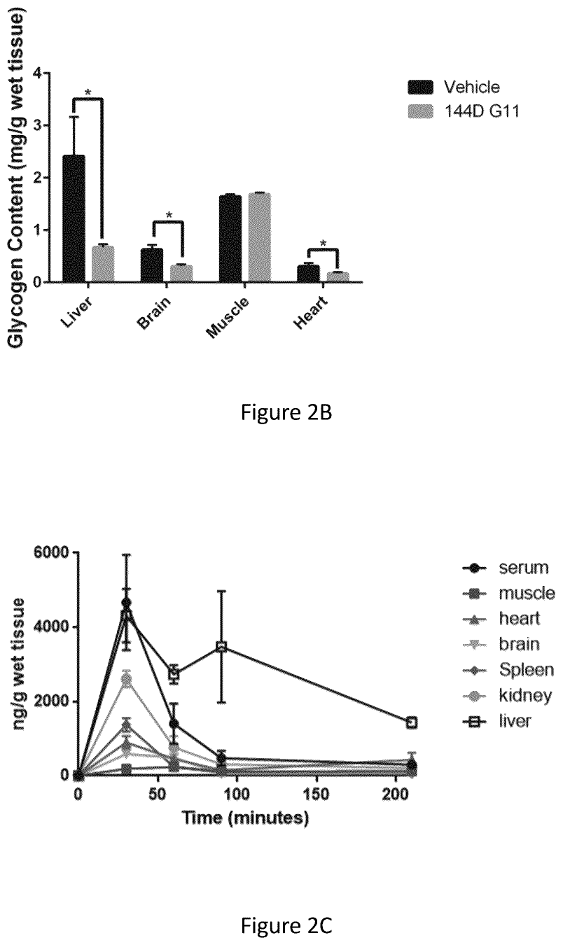

The presented experiments combine in vivo, ex vivo and in vitro studies on the therapeutic potential of the newly discovered compound Compound 1 for treating APBD. In the in vivo section, the inventors have tested Compound 1 for its capacity to correct disease phenotypes in Gbe^{ys/ys} female mice. Two arms, 5% DMSO vehicle and Compound 1, of initially n=7-9 animals each were used. These numbers were demonstrated retrospectively to provide sufficient power because, based on the means and SD obtained, a power of 80% is already attained at n=5 animals/arm. Additional open field, gait, and extension reflex tests (FIGS. 1E-1H) also included a C57BL/6 wild type control arm of n=9 animals. Animals were excluded from the experiment if weight was reduced by >10% between sequential weightings or by >20% from initiation. Sample size was slightly reduced over time due to death. 150 μL of 250 mg/kg Compound 1 in 5% DMSO were injected twice a week. Vehicle control was 5% DMSO. Injection was intravenous (IV) for the first month, followed by subcutaneous (SC) injection due to lack of injection space and scaring in animal tails. The inventors initiated the injections either at the age of 4 months, two months prior to disease onset, assuming a preferred prophylactic effect, or at the 6 months age of onset for comparison. Treatment was continued until the age of 10 months. The effect of Compound 1 on various motor parameters was tested approximately every two weeks. At the end of these experiments, some of the mice were sacrificed by cervical dislocation and tissues from n=2 wild type, n=7 Gbe^{ys/ys} vehicle-treated, and n=9 Compound 1-treated mice were collected, sectioned, fixed, and stained for diastase-resistant PG by PAS (FIGS. 2A-2C). Tissue glycogen was determined biochemically as described. In addition, Compound 1 pharmacokinetic profile was determined by LC-MS/MS in serum and tissues derived from n=3 mice/time point. Experimenters were blinded to treatment allocation.

Ex vivo studies were done in APBD patient-derived skin fibroblasts and in liver sections from Gbe^{ys/ys} mice, as liver had the highest PG levels. In vitro studies were conducted in cell lysates.

### Histological PG and Glycogen Determination

Brain, heart, muscle, nerve fascicles (peripheral nerves), and liver tissues from wt and Compound 1 and vehicle treated Gbe^{ys/ys} animals were separated to characterize the histopathological effects of Compound 1. Tissues were extracted, fixed, embedded in paraffin, and sectioned. After deparaffinization, sections were treated for 5 min with 0.5% diastase to digest non-polyglucosan glycogen, leaving behind polyglucosan. Sections were then washed, stained for polyglucosan with PAS and counterstained with hematoxylin, and analyzed by light microscopy, all as previously described. For biochemical glycogen determination, 100 mg of each tissue was subjected to alkaline hydrolysis and boiling followed by ethanol precipitation of glycogen. Glycogen was then enzymatically digested to glucose by amyloglucosidase (Sigma). Following digestion, total glycogen was determined based on the glucose content using the Sigma GAGO20 kit.

### Imaging and Image-Based Phenotyping

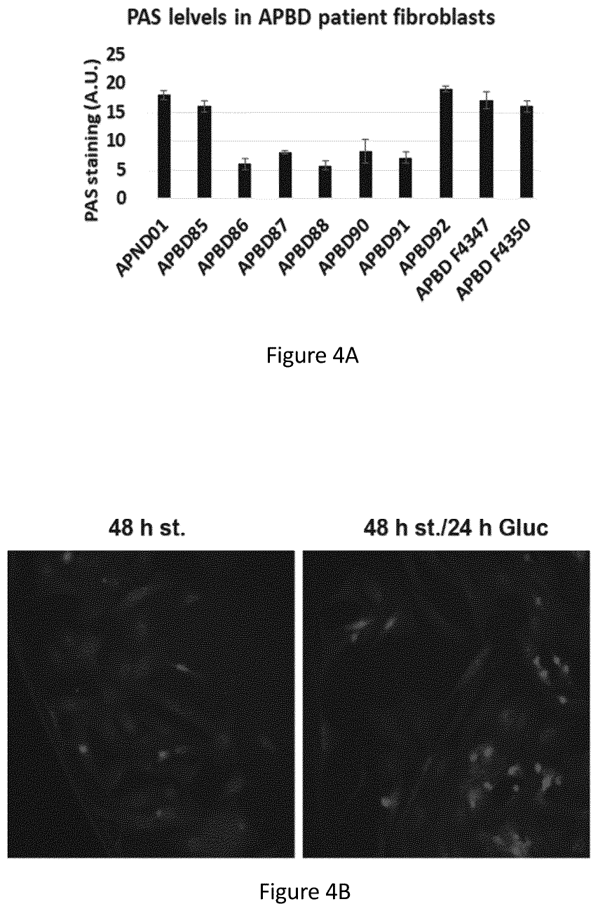

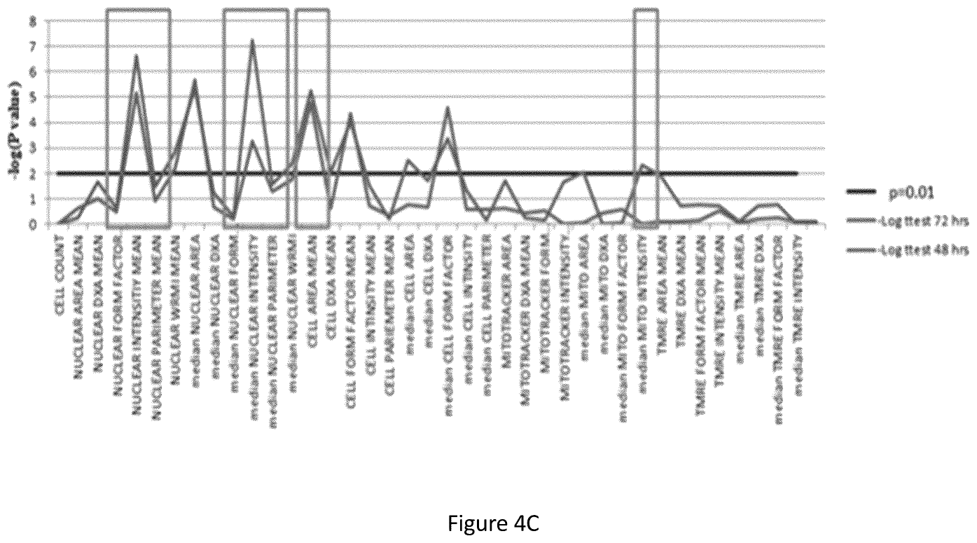

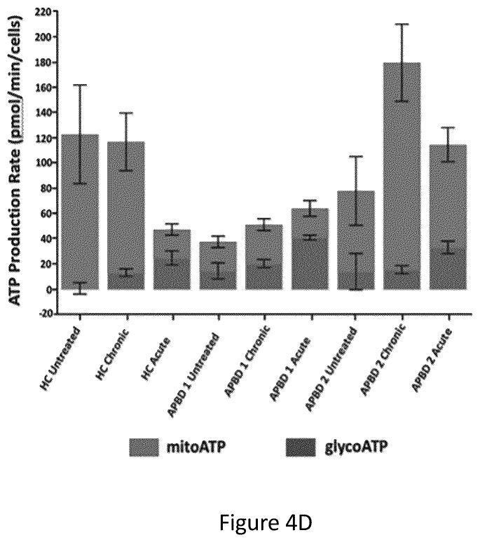

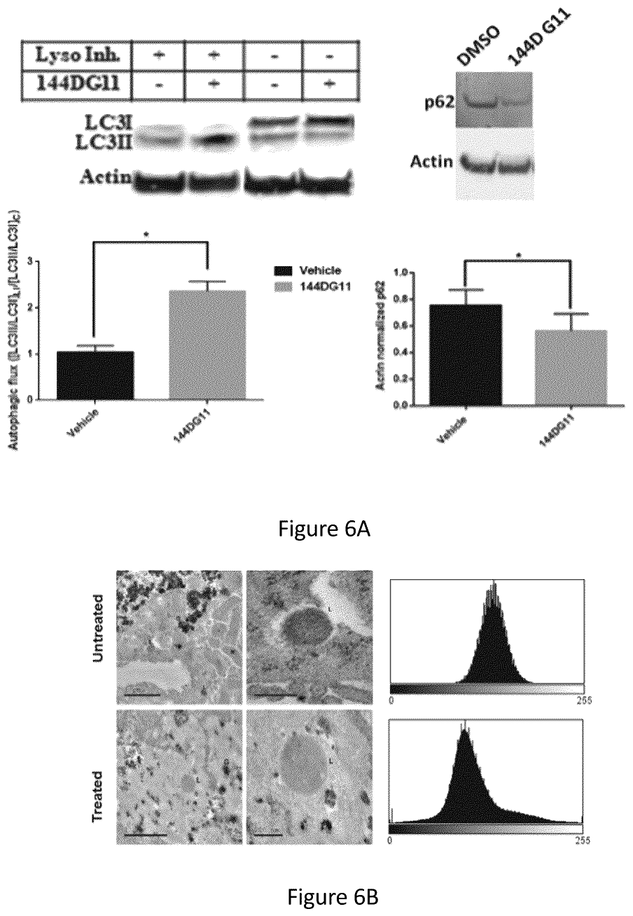

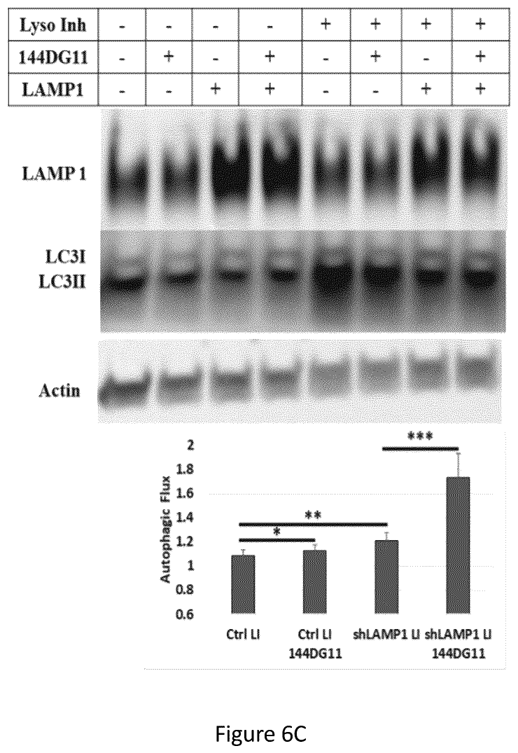

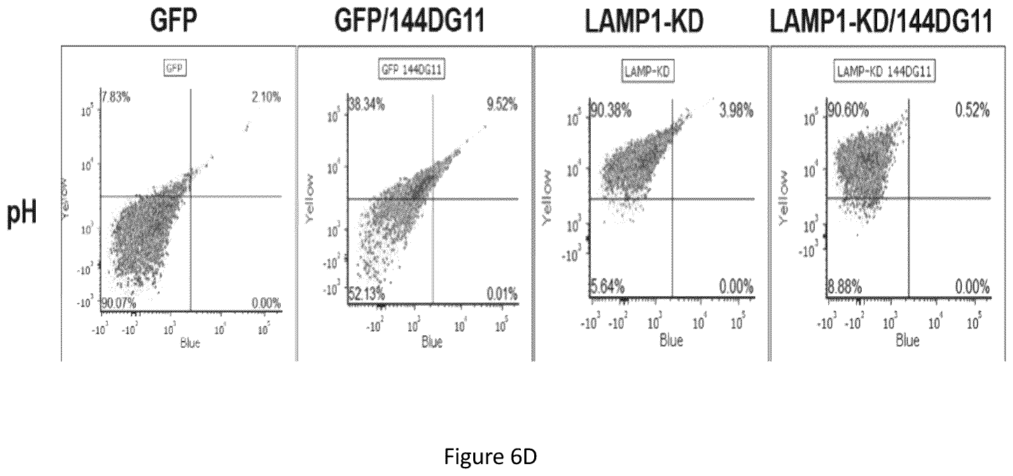

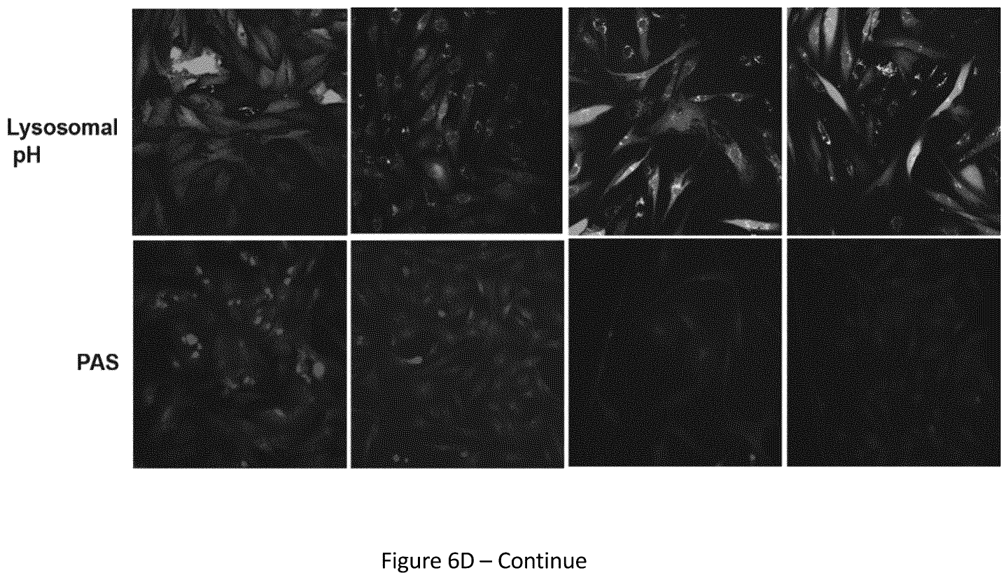

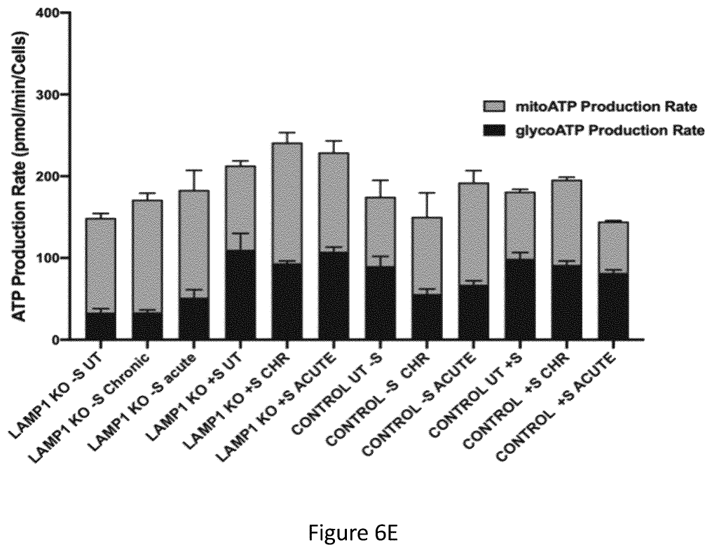

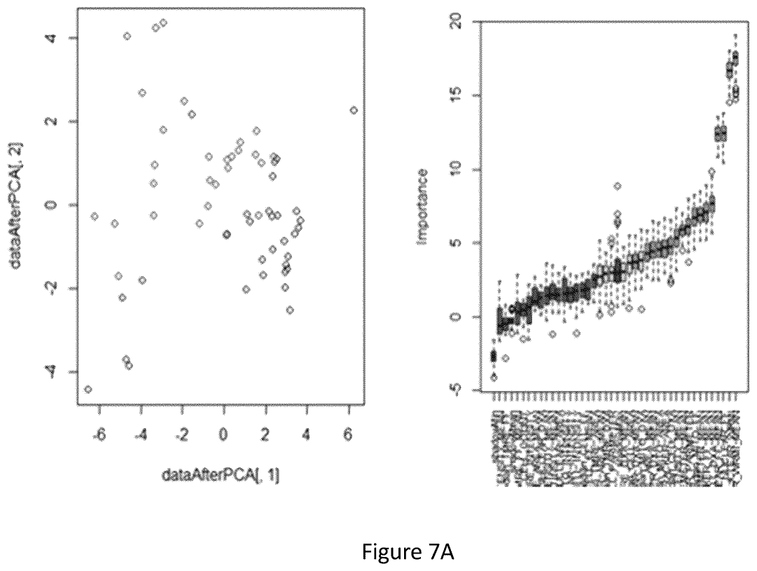

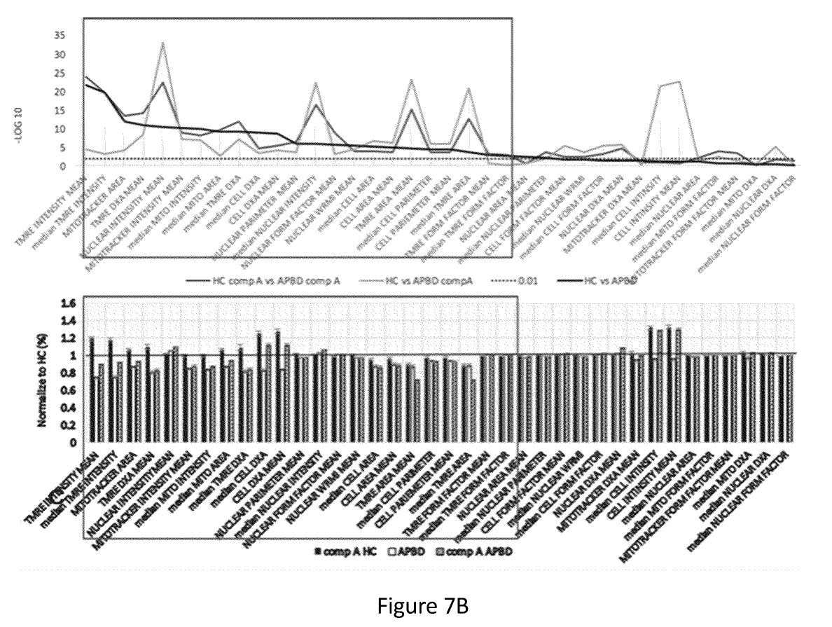

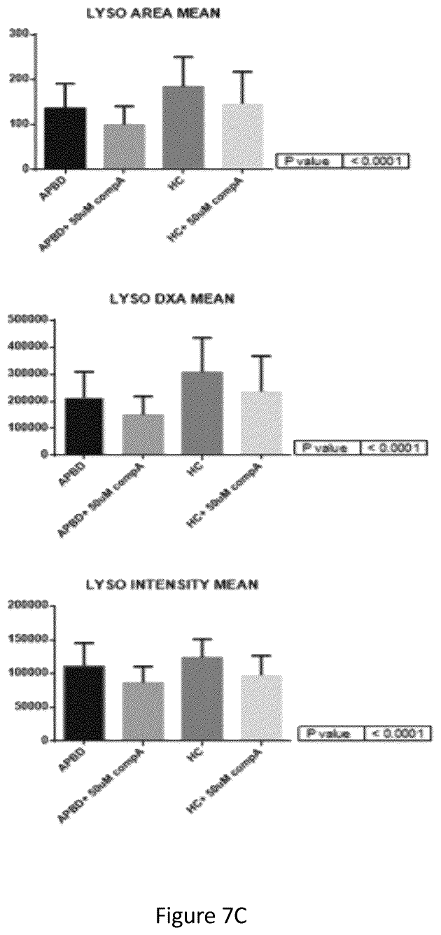

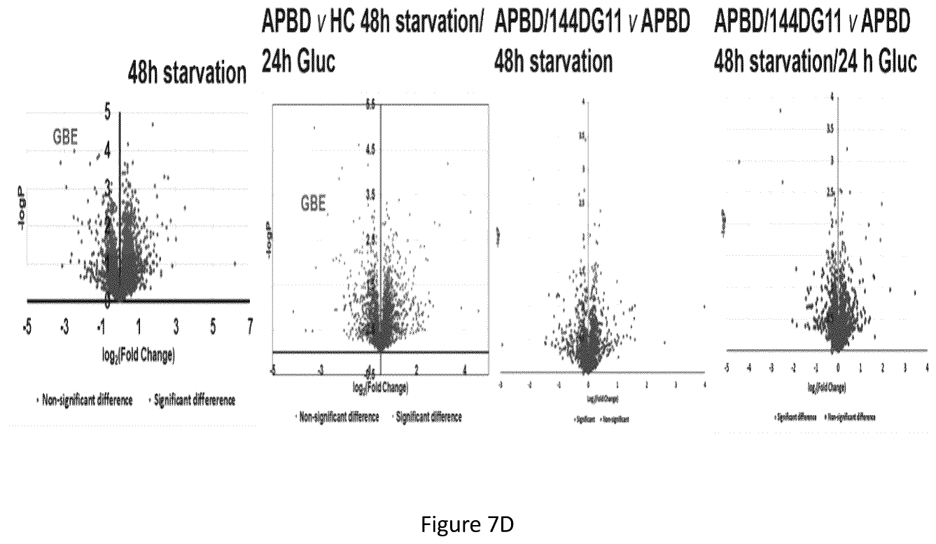

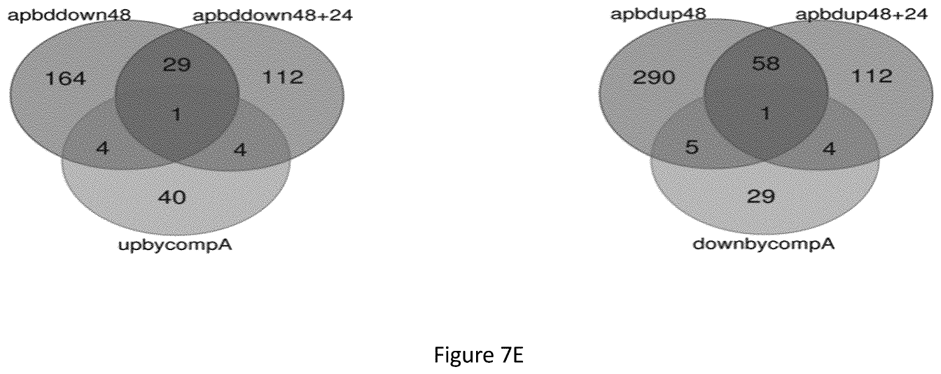

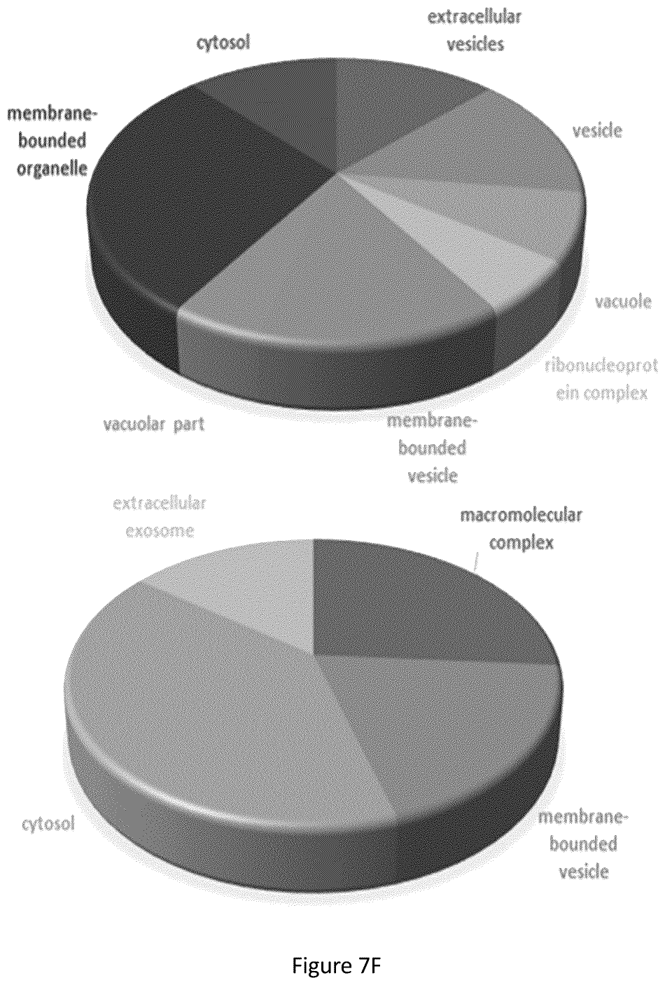

APBD skin fibroblasts were seeded at 1,000 cells/well and cultured in specialized microscopy-grade 96-well plates (Grenier Bio-One, Germany). Following the different treatments, a mix of Thermo Scientific cellular fluorescent dyes in PBS was added to each well for 30 min at 37° C. in a 5% CO₂ incubator. This mix (FIGS. 4C and 7B) included DAPI (1 μg/ml, nuclear (DNA) stain), MitoTracker Green (500 nM, potential-independent mitochondrial stain), TMRE (500 potential-dependent mitochondrial stain), and Cell Mask Deep Red (0.5 μg/ml, cytosol stain). In FIG. 7C, only lysosomes were stained with LysoTracker Deep Red (75 nM). Cells were then fixed with 4% paraformaldehyde (PFA), washed with PBS and plates were transferred to an InCell2200 (GE Healthcare, U.K.) machine for image acquisition at 40× magnification. The output produced was based on comparative fluorescence intensity. Object segmentation was carried out using multi-target analysis in the GE analysis workstation to identify the nuclei and cell boundary. All the assay parameters (including the acquisition exposure times, objective, and the analysis parameters) were kept constant for all assay repetitions. For PAS staining of glycogen (FIGS. 4 and 6C), fixed cells were washed with PBS, permeabilized with 0.1% Triton X-100, washed again stained and then imaged.

### Pharmacokinetics

For pharmacokinetic analysis, 100 μL serum as well as brain, kidney, hind limb quad muscle, heart, liver, and spleen tissues were collected, homogenized, and extracted with acetonitrile following established guidelines. Calibration curves were made with 0, 1, 10, 100, and 1,000 ng/ml Compound 1 in 1 mg/ml solutions of 4-tert-butyl-2-(4H-1,2,4-triazol-4-yl)phenol (ChemBridge) as internal standard (IS). Tissue samples were then dissolved in 1 mg/ml IS solutions and spiked with 0-1,000 ng/ml Compound 1 to generate standard curves from which tissue levels of Compound 1 were determined. Samples were analyzed by the LC-MS/MS Sciex Triple Quad™ 5500 mass spectrometer.

### Ethics

In vivo work was approved by the Hebrew University IACUC.

### Statistical Analysis

In FIGS. 1A-1M the significance of overall trends was tested by Two-way ANOVA with repeated measures. This test determines how a response is affected by two factors: Compound 1 v control, which is given repeatedly (hence repeated measures) and duration of administration. The Bonferroni test was used to compare between Compound 1 and vehicle in a way which corrects for the multiple comparisons and is therefore very robust (since the threshold for determining significance at each time point is reduced in a manner inversely proportional to the number of comparisons). Consequently, most differences at specific time points became insignificant due to the increase in the number of comparisons and sometimes the inventors chose to also show the data of multiple t-tests which do not correct for multiple comparisons. In FIGS. 4D and 6E the inventors used One Way ANOVA with Sidak's post-hoc correction for multiple comparisons. Other statistical tests used were Student t-tests.

### Target Identification by Nematic Protein Organization Technique (NPOT)

NPOT® was applied on human heathy fibroblasts and fibroblasts from two APBD patients. All the analyses were done by Inoviem Scientific Ltd. in a blind manner. Protein homogenates from dry pellets of these fibroblasts were prepared by three cycles of fast freezing (liquid nitrogen) and slow thawing (on ice) and mixed at a maximal vortex speed for 30 seconds. Sample protein concentration was 50-66 mg/ml as determined by the BCA method. NPOT® is a proprietary technology offered by Inoviem Scientific dedicated to the isolation and identification of specific macromolecular scaffolds implemented in basic conditions or in pathological situations directly from human tissues. The technology is based on Kirkwood-Buff molecular crowding and aggregation theory. It enables the formation and label-free identification of macromolecular complexes involved in physiological or pathological processes. The particular strength of Inoviem Scientific is the ability to analyze drug-protein and protein-protein interactions directly in human tissue, from complex mixtures without disrupting the native molecular conformation, consequently remaining in initial physiological or pathological condition.

Under laminar flow and sterile conditions, 10⁻⁶ M of compounds Compound 1 and a negative control from the HTS screen were mixed separately with the protein homogenates (containing soluble and membrane proteins) and subjected to NPOT® isolation. The macromolecular assemblies associated with the ligand are separated using a differential microdialysis system, wherein the macromolecules (protein groups) migrate in the liquid phase based on their physico-chemical properties. The migrating macromolecules gradually grow from nematic crystals to macromolecular heteroassemblies thanks to the molecular interactions between the tested drug and its targets. The heteroassemblies were left overnight and isolated in a 96-well plate prior to identification by LC-MS/MS.

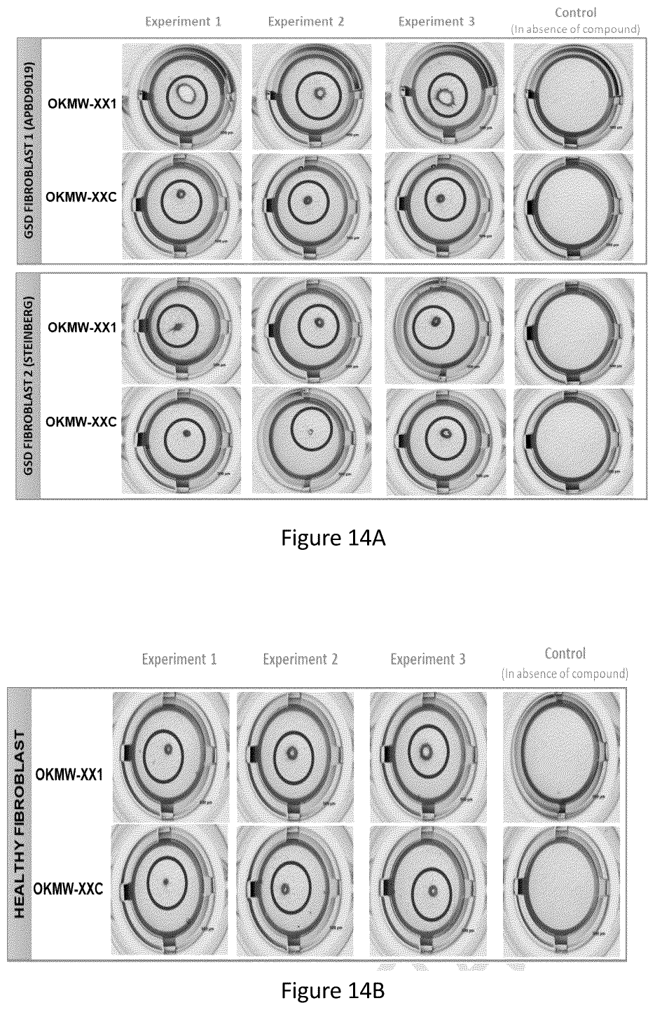

The formed heteroassemblies in presence of Compound 1 and the negative control in APBD-patients and HC fibroblasts are shown in FIGS. 14A-14B. Each compound in contact with the indicated protein homogenates gave rise to clearly defined hetero assemblies with common reticular morphology. The experiments were done in triplicate for each compound. For each of these biological replicates, heteroassemblies were isolated and their protein content analyzed by LC-MS/MS. The negative control is obtained with the protein homogenate in the NPOT® conditions without the addition of compound and does not present any aggregation. This further confirms that the formation of the heteroassembly is initiated by the compounds, and not by an endogenous small molecule, through their interactions with primary targets.

Under a Zeiss microscope SteREO Discovery V8, each formed heteroassembly was isolated by microdissection and washed in acetone prior to solubilization in standard HBSS solution. Solubilized proteins were filtered through a 4-15% mini-PROTEAN gel. After migration, the gel was coloured with a colloidal blue solution in order to visually estimate the number of proteins present in the gel, and the relative quantity of proteins to use for the following digestion step and injection in the LC-MS/MS instrument for proteomics analysis.

Proteomics was outsourced to the “Laboratoire de Spectrometrie de Masse Bio-Organique” (LSMBO) from the UMR 7178. Heteroassemblies were solubilized directly in 10 μL of 2D buffer (7 M Urea, 2 M Thiourea, 4% CHAPS, 20 mM DTT, 1 mM PMSF). Proteins were precipitated in acetate buffer and centrifuged for 20 minutes at 7,500 g. Thereafter pellets were digested for 1 hour with Trypsin Gold (Promega) at 37 ° C. Trypsin Gold was resuspended at 1 μg/μL in 50 mM acetic acid, then diluted in 40 mM NH₄HCO₃ to 20 μg/mL. The samples were dried in Speed Vac® at room temperature. Peptides were purified and concentrated by using ZipTip® pipette tips (Millipore Corporation) before proceeding for mass spectrometry analysis through 1-hour nano-LC-MS/MS analyses protocol in an ESI-QUAD-TOF machine. Proteins were identified using Mascot software (Rank=1, score=25, minimal length=6 amino-acids, FDR=1%). For peptide mapping, the following database was used: -HumaniRTUN_DCpUN_JUS Bank (for human samples).

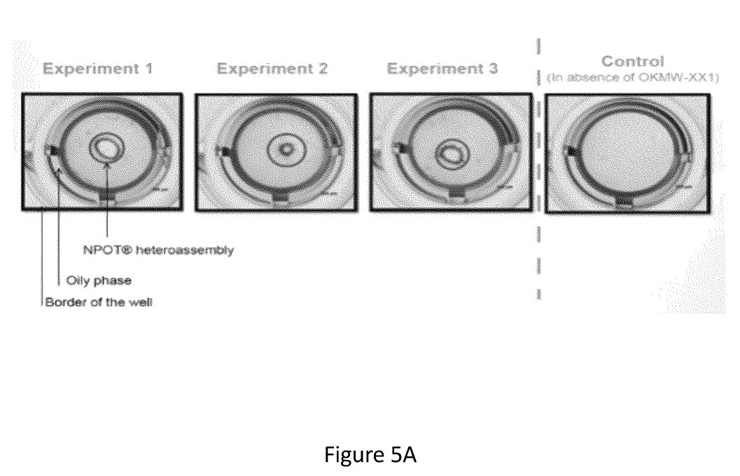

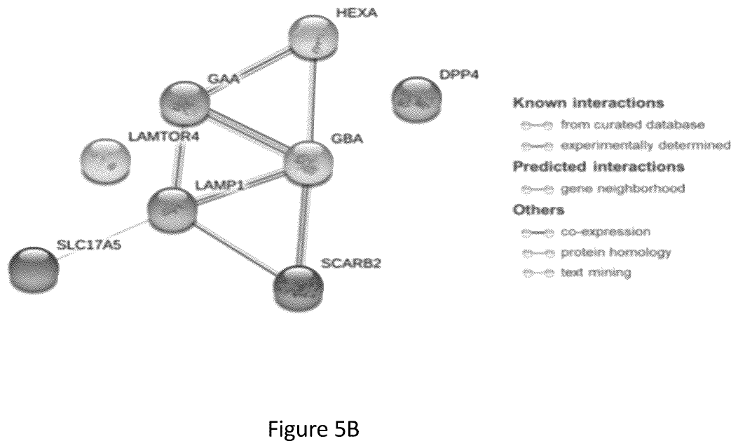

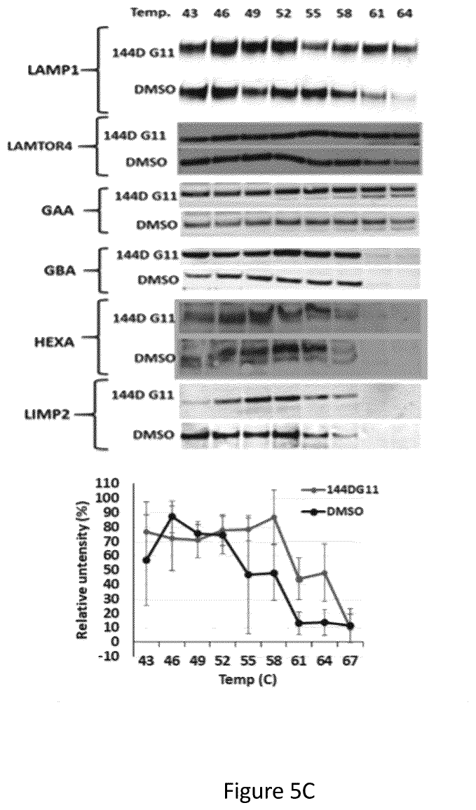

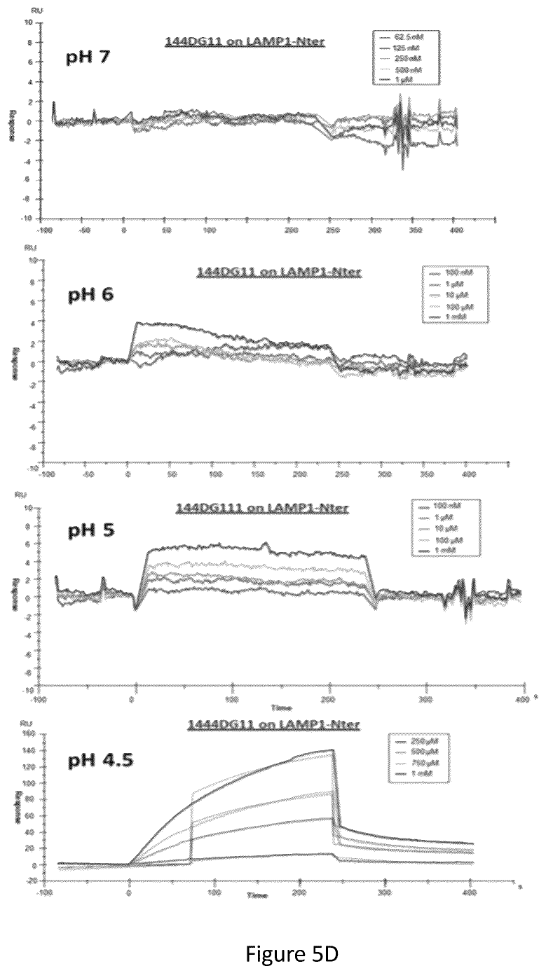

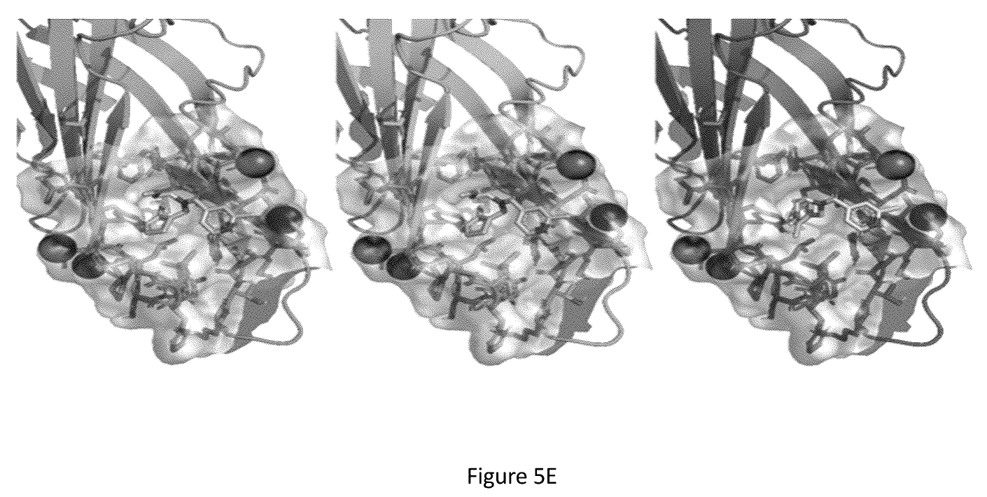

For data analysis and target deconvolution Inoviem Scientific developed its own database and software to allow an accurate and robust analysis of the proteins present in NPOT® datasets and simplify proteins ranking while removing protein contaminants. Inoviem Protein Ranking and Analysis (InoPERA®) database comprises all the NPOT® datasets obtained on various tissues, organs or cell lines, varied species, and unrelated chemical compounds. InoPERA® software is then able to calculate the occurrence of one given gene in the entire database, or specific datasets matching defined criteria of species, organs etc. Inoviem removed contaminants that have been observed in NPOT® performed in human tissues and cells, which correspond to 613 NPOT® coupled LC-MS/MS analyses. Consequently, this tool is able to quickly highlight rare proteins within a dataset that would make new therapeutic targets (FIG. 5B).

Another bioinformatics resource—DAVID was also used to find tissue-specific expression, gene-ontology, and functional-related gene group enrichment. Network enrichment within a dataset was investigated using STRING analysis (string-db.org). STRING is one of the core data resource of ELIXIR (as Ensembl or UniProt are) which contains known and predicted protein-protein interactions. Inoviem has used the stringent parameters, keeping only the known interactions (“experimentally determined” and “curated databases” interaction sources). This allowed deciphering the protein-protein associations within a complex dataset, which further completed the DAVID pathway analysis. In addition, Reactome (reactome.org)—a free, open-source, curated and peer-reviewed pathway database was used. This database provides intuitive bioinformatics tools for the visualization, interpretation, and analysis of pathway knowledge to support findings obtained elsewhere.

In the bioinformatic pipeline, the first step of filtering consisted of removing the mass spectrometry “false positives”, i.e., the proteins found in one replicate and with only one specific peptide. Then, the datasets were compared in a 2 by 2 matrix (144DG11 and its respective negative control) in human skin fibroblast tissue. The next step of protein list analysis was identification of non-specific proteins, i.e., proteins that are found in a recurrent manner in all NPOT® experiments (InoPERA®). Contaminants (or “frequent hits”) observed in human skin fibroblasts were removed. Cleared proteins lists of the interactome thus represent potential specific targets for Compound 1. Using this pipeline 28 proteins were found to interact specifically with Compound 1. Compound 1 interactome's specific protein lists were then analyzed independently by DAVID to find tissue-specific expression, gene-ontology, and functional-related gene group's enrichment. The main canonical and disease and function pathways underlying the Compound 1 interactome were the lysosomal membranes (reference: GO:0005765 and KEGG pathway hsa04142). In parallel, STRING analysis (string-db.org) was used to visualize prominent nodes and enriched networks. For this first ranking of the compound interactome's specific proteins, the inventors did not use the signal intensity of the peptides sequenced by MS because 1) the intrinsic properties of the technology cannot be based on protein quantitation (conversely to classic immunoprecipitation protocol for example), and 2) the inventors do not use LC-MS/MS quantitative protocols (which would imply higher cost and longer time analysis). This unbiased analysis allowed Inoviem to classify potential, relevant proteins and categorize them according to their involvement in specific pathways, or in relation with specific diseases. Following this bioinformatic selection, 8 proteins belonging to the autophagosomal-autolysosomal pathway were discovered (FIG. 5B). The discovery of this well-defined and enriched network demonstrates the overall success of the NPOT® experiment.

### Computational Docking Analysis

LAMP1 is divided into five domains: (1) residues M1 -A28: signal sequence; (2) residues A29-R195: N-terminal domain; (3)residues P196-S216: linker between the domains; (4) residues 5217-D378: C-terminal domain; and (5) residues E379-I417: the transmembrane segment. The inventors have analyzed only the N- and the C-terminal domains since: 1. The signal sequence and the transmembrane segment are assumed to be irrelevant for the binding of small molecules; and 2. The linker between the domains is unstructured and heavily glycosylated (7 out of 20 residues) and thus, too complicated to model. The inventors have not considered glycosylation in the N- and the C-terminals. The C- and N-Terminal domains were modeled based on the known crystal structure of mouse LAMP1 C-terminal domain (PDB ID 5gv0) which is structurally highly similar to the N-terminal domain. The MODELLER software tool was used for homology modeling, producing 5 optional models for each domain. The obtained 10 models (as well as 5gv0 itself) were prepared in pH 5 by the “protein preparation wizard” as implemented in Schrodinger 2020-2. Possible binding sites were identified by three different computational tools: SiteMap, FtSite and fPocket. Overall, 130 optional sites were identified in 11 LAMP1 3D structures. Docking computations were performed for each of the putative binding sites: 418 out of a large and diverse database of ˜30 million molecules were chosen as decoys according to Compound 1 applicability domain (Lipinski rules properties). The decoys library was narrowed down to 233 based on chemical similarity (Tanimoto coefficient >=0.7). Docking computations for Compound 1 in a set of molecules composed of Compound 1 and these 233 decoys (prepared in pH 5) were performed for every putative binding site in every model (overall 130 sites). The computations were performed using the Glide algorithm, as implemented in Schrodinger 2020-2. According to the docking results analysis, in 18 out of 130 sites Compound 1 was ranked at the top 10%: 3 grids from SiteMap, 3 from FtSite, and 12 from fPocket. 8 grids were in the C-terminal domain and 10 in the N-terminal domain.

The inventors noticed that according to one of the models of the N-terminal domain (Model #4), Compound 1 was ranked in the top 10% for 6 out of these 18 sites. Analyzing the results, the inventors realized that site 1 of SiteMap, site 3 of fPocket, and site 2 of FtSite refer to the same pocket (residues F50-D55, N62, L67, F118, Y120-L122, T125, L127-S133, N164-V166).

The inventors examined the differences between the three binding modes (FIG. 5E): It seems that two out of three binding modes (SiteMap and fPocket) are identical and that in the third one (FtSite) part of Compound 1 went through a rotation relative to the other two.

To predict the probability of obtaining a unique binding as observed for Compound 1 only by chance, the inventor repeated the analysis presented above for Compound 1 for all the 233 decoys. Only in 14 out of 234 molecules (233 decoys +Compound 1), the inventors observed the same results as for Compound 1—i.e., a molecule the binding of which to a pocket was predicted by 3 different tools (Table 1). This indicates that the chances are relatively small (14/234 ˜6%). Moreover, the pocket identified for Compound 1 (FIG. 5E) is the most common (matched 5 molecules out of 14, Table 1). This indicates that this pocket may be druggable and bind a relatively high number of compounds, which is an advantage for putative medicinal chemistry improvement of Compound 1. The table shows cases in which according to 3 different tools (for predicting binding sites), a molecule enters to the same pocket. The 3 digits following “site” in the first column indicate the site ranking by SiteMap (first number), FtSite (second number), and fPocket (third number).

TABLE 1
Molecules predicted to the same pocket by 3 different tools.

| | | Number of |
|---|---|---|
| Model, Site ranking | Compounds (InchiKeys) | compounds |
| 5gv0, site112 | ISNBQFVIRRXMNN-UHFFFAOYSA-N | 1 |
| HN.B99990001, | SDIOOFTYDJNAOO-UHFFFAOYSA-N | 1 |
| site113 | | |
| HN.B99990004, | 144DG11 (COMPOUND 1) | 5 |
| site123 | AFRQZZBRQMVSOV-UHFFFAOYSA-N* | |
| | OYOBHSNWLONJLP-UHFFFAOYSA-N | |
| | ULMGNNFJGOAZQX-UHFFFAOYSA-N | |
| | VODWQUHDWRIEOI-UHFFFAOYSA-N | |
| HN.B99990005, | LHKJBSUPOYJYCL-UHFFFAOYSA-N | 1 |
| site121 | | |
| HN.B99990005, | UIVJRUWFXCGSSM-UHFFFAOYSA-N | 2 |
| site233 | XDGVKHPNDVKOPJ-UHFFFAOYSA-N | |
| HC.B99990001, | OHHMZSKNHGGUAX-UHFFFAOYSA-N | 1 |
| site223 | | |
| HC.B99990002, | CKHGBJKQAHAIDZ-UHFFFAOYSA-N | 3 |
| site 131 | GLXDFBGZVSREGI-UHFFFAOYSA-N | |
| | WTTMQZWWJFRCSI-UHFFFAOYSA-N | |
| HC.B99990003, | AFRQZZBRQMVSOV-UHFFFAOYSA-N* | 1 |
| site233 | | |

*This molecule binds to two different binding sites

The inventors repeated the analysis in a less restricted definition of the binding site and obtained similar results: 45 out of the 234 molecules (˜19%) were docked successfully to at least one of the predicted pockets. However, in 14 out of the 45 molecules, the inventors observed binding to more than one site, which indicates promiscuity. Therefore, overall, 31 molecules out of 234 were docked successfully to one of the putative sites (˜12.7%). In summary, the inventors have computationally identified a possible binding site for Compound 1 in the N-terminal domain of LAMP1 and predict with high confidence that this result is specific for Compound 1 since the probability to obtain the same results for decoy molecules is low.

### Transmission Electron Microscopy (TEM)

Liver tissue was minced and fixed in a solution containing 2% paraformaldehyde, 2.5% glutaraldehyde (EM grade) in 0.1 M sodium cacodylate buffer pH 7.3 for 2 hours at RT, followed by 24 h at 4 ° C. Tissue was then washed 4 times with sodium cacodylate and postfixed for 1 h with 1% osmium tetroxide and 1.5% potassium ferricyanide in sodium cacodylate. Then sample was washed 4 times with the same buffer and dehydrated with graded series of ethanol solutions (30, 50, 70, 80, 90, 95%) for 10 minutes each and then 100% ethanol 3 times for 20 minutes each. Subsequently, samples were treated with 2 changes of propylene oxide. Samples were then infiltrated with series of epoxy resin (25, 50, 75, 100%—24 h in each) and polymerized in the oven at 60° C. for 48 hours. The blocks were sectioned by an ultramicrotome (Ultracut E, Riechert-Jung) and obtained sections of 80 nm were stained with uranyl acetate and lead citrate. Sections were observed by Jeol JEM 1400 Plus Transmission Electron Microscope and images were taken using Gatan Orius CCD camera.

### Proteomics (FIG. 7)

Sample preparation for MS analysis. Cell lysates in RIPA buffer containing protease inhibitors were clarified by centrifugation and 40 ρg of protein was used for protein precipitation by the chloroform/methanol method. The precipitated proteins were solubilized in 100 μl of 8 M urea, 10 mM DTT, 25 mM Tris-HCl pH 8.0 and incubated for 30 min at 22 ° C. Iodoacetamide (55 mM) was added, and samples were incubated for 30 min (22 ° C., in the dark), followed by addition of DTT (10 mM). Fifty μl of the samples was transferred into a new tube, diluted by the addition of 7 volumes of 25 mM Tris-HCl pH 8.0 and sequencing-grade modified Trypsin (Promega Corp., Madison, WI) was added (0.35 μg/sample) followed by incubation overnight at 37° C. with gentle agitation. The samples were acidified by addition of 0.2% formic acid and desalted on C18 home-made Stage tips. Peptide concentration was determined by Absorbance at 280 nm and 0.75 μg of peptides were injected into the mass spectrometer.

Nano LC-MS/MS analysis. MS analysis was performed using a Q Exactive-HF mass spectrometer (Thermo Fisher Scientific, Waltham, MA USA) coupled on-line to a nanoflow UHPLC instrument, Ultimate 3000 Dionex (Thermo Fisher Scientific, Waltham, MA USA). Peptides dissolved in 0.1% formic acid were separated without a trap column over a 120 min acetonitrile gradient run at a flow rate of 0.3 μl/min on a reverse phase 25-cm-long C18 column (75 μm ID, 2 μm, 100 Å, Thermo PepMapRSLC). The instrument settings were as previously described. Survey scans (300-1,650 m/z, target value 3E6 charges, maximum ion injection time 20 ms) were acquired and followed by higher energy collisional dissociation (HCD)-based fragmentation (normalized collision energy 27). A resolution of 60,000 was used for survey scans and up to 15 dynamically chosen most abundant precursor ions, with “peptide preferable” profile were fragmented (isolation window 1.6 m/z). The MS/MS scans were acquired at a resolution of 15,000 (target value 1E5 charges, maximum ion injection times 25 ms). Dynamic exclusion was 20 sec. Data were acquired using Xcalibur software (Thermo Scientific). To avoid a carryover, the column was washed with 80% acetonitrile, 0.1% formic acid for 25 min between samples.

MS data analysis. Mass spectra data were processed using the MaxQuant computational platform, version 1.6.14.0. Peak lists were searched against the Uniprot human FASTA sequence database from May 19, 2020, containing 49,974 entries. The search included cysteine carbamidomethylation as a fixed modification, N-terminal acetylation and oxidation of methionine as variable modifications and allowed up to two miscleavages. The match-between-runs option was used. Peptides with a length of at least seven amino acids were considered and the required FDR was set to 1% at the peptide and protein level. Relative protein quantification in MaxQuant was performed using the label-free quantification (LFQ) algorithm. Statistical analysis (n=4-7) was performed using the Perseus statistical package. Only those proteins for which at least 3 valid LFQ values were obtained in at least one sample group were accepted for statistical analysis by t-test (p <0.05).

### EXAMPLE 1

### Compound 1 Improves Survival and Motor Deficiencies in Gbe^{ys/ys} Mice

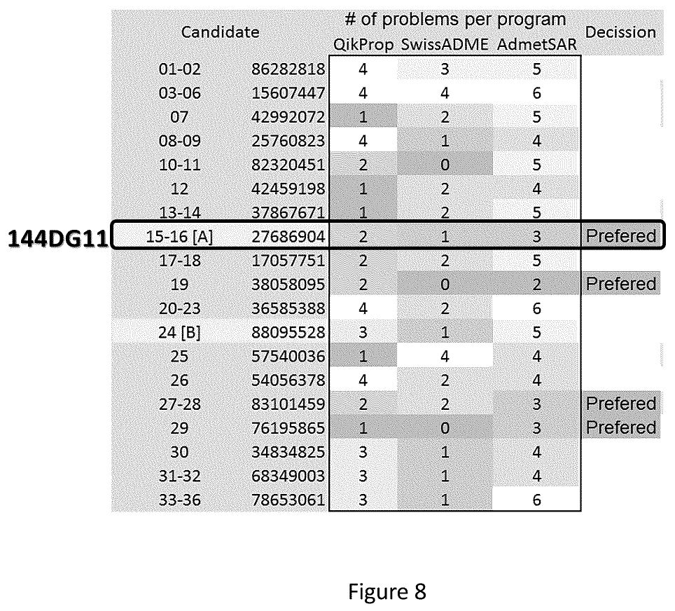

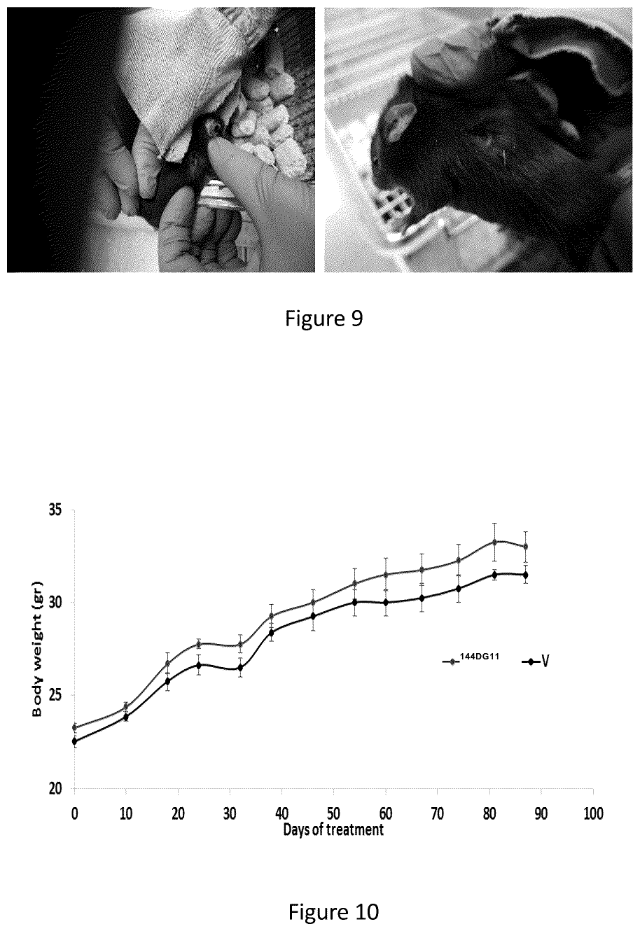

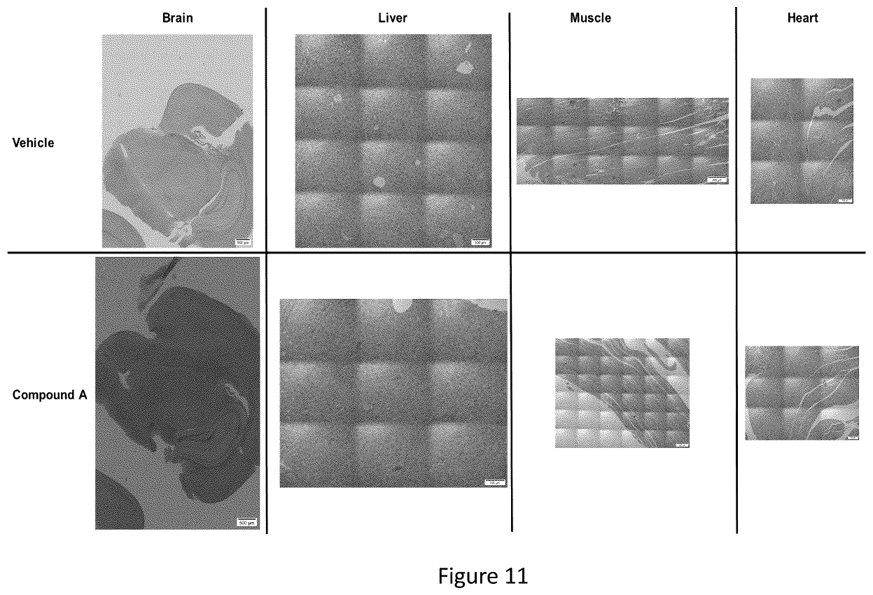

The inventors have tested Compound 1 (FIG. 1A) for its capacity to correct the deficient motor phenotypes and short lifespan in the APBD mouse model Gbe^{ys/ys}. Compound 1 is one of 19 PG reducing HTS hits previously discovered by the inventors. It was selected by in silico ADMET (Absorption, Distribution, Metabolism, Excretion, and Toxicity) tests run on these hits to predict which of them should be safe and pharmacokinetically and pharmacodynamically preferred and is therefore worth further pursuit (FIG. 8, compound “A”). Indeed, low ADMET scoring compounds such as “B” (FIG. 8), were not efficacious and caused adverse effects such as wounds (FIG. 9). Moreover, safety assessment in wild type mice confirmed that, administered for 3 months at 250 mg/kg in 5% DMSO (the highest dose possible due to solubility and DMSO toxicity issues), Compound 1 did not influence animals' weight gain over time (FIG. 10). The compound also did not produce any histopathological damage or lesions in brain, liver, skeletal muscle, and heart after 3 months exposure (FIG. 11). Following 1 h and 24 h treatments, mice were also examined for abnormal spontaneous behavior, such as immobility, excessive running, stereotyped movements, and abnormal posture (Irwin tests). Compound 1 did not cause any adverse effect in these Irwin tests (Table 2).

TABLE 2
Irwin test results of Compound 1.

| | | 50 mg/kg | 250 mg/kg | 50 mg/kg | 250 mg/kg |
|---|---|---|---|---|---|
| | Vehicle | 1 h | 1 h | 24 h | 24 h |
| Coat color | Black | Black | Black | Black | Black |
| Presence of | 3 | 3 | 3 | 3 | 3 |
| whiskers | | | | | |
| Appearance | 2 | 2 | 2 | 2 | 2 |
| of fur | | | | | |
| Piloerection | 0 | 0 | 0 | 0 | 0 |
| Patches of | 0 | 0 | 0 | 0 | 0 |
| missing fur | | | | | |
| on face | | | | | |
| Patches of | 0 | 0 | 0 | 0 | 0 |
| missing fur | | | | | |
| on body | | | | | |
| Wounds | 0 | 0 | 0 | 0 | 0 |
| Transfer | 5 | 5 | 5 | 5 | 5 |
| behavior | | | | | |
| Body | 3.5 | 3.5 | 3 | 3.5 | 3 |
| position | | | | | |
| Tremor | 0 | 0 | 0 | 0 | 0 |
| Gait | 0 | 0 | 0 | 0 | 0 |
| Pelvic | 2 | 2 | 2 | 2 | 2 |
| elevation | | | | | |
| Tail | 1 | 1 | 1 | 1 | 1 |
| elevation | | | | | |
| Touch | 2 | 2 | 2 | 2 | 2 |
| escape | | | | | |
| Positional | 0 | 0 | 0 | 0 | 0 |
| passivity | | | | | |
| Trunk curl | 0 | 0 | 0 | 0 | 0 |
| Righting | 0 | 0 | 0 | 0 | 0 |
| reflex | | | | | |
| Salivation | 0 | 0 | 0 | 0 | 0 |
| Extension | 2 | 2 | 2 | 2 | 2 |
| reflex | | | | | |

Importantly, as FIG. 1B shows, treatment with Compound 1 has significantly improved animal survival (log-rank test p-value <0.000692) as compared to vehicle treated animals. Lifespan extension probably mirrors improvement of several parameters related to animals' ability to thrive. The most prominent parameter in that respect is animal weight. Compound 1 has indeed mitigated the decline in animal weight over time caused by the disease (FIG. 1C).The inventors have also tested every two weeks the effect of Compound 1 on various motor parameters. Compound 1 has improved open field performance (FIG. 1D) from a relatively advanced stage of disease progression (8 months, 134 days post injection (FIG. 1D). These improvements were manifested as increased locomotion and an increased tendency to move towards the center (FIG. 1E), perhaps also associated with amelioration of stress and anxiety. The progressive deterioration of Gbe^{ys/ys} mice in open field performance is related to their gait deficiency. Therefore, the inventors tested the effect of Compound 1 on gait at the mouse age of 9 months when gait is severely affected. At that age Compound 1 has indeed improved gait, or increased stride length (FIG. 1F). The data also show that, of all motor parameters tested, the most pronounced ameliorating effect was on the overall extension reflex (FIG. 1G). Overall extension reflex throughout the study period, was significantly improved by Compound 1 (FIG. 1G, p<0.05) as it was at 9 specific time points (asterisks in FIG. 1G). This effect is especially important since its patient correlate is pyramidal tetraparesis, or upper motor neuron signs, which are one of the main neurological deficiencies in APBD patients. Importantly, while open field performance (FIG. 1E), gait (FIG. 1F) and extension reflex (FIG. 111) were significantly improved by Compound 1, they were not restored to wild type levels, demonstrating that while efficacious, Compound 1 performance still leaves some room for future improvement.

For studying the effects of Compound 1 on motor parameters, the inventors initiated the injection of Compound 1 at the age of 4 months, two months prior to disease onset, assuming a preferred prophylactic effect. Such an effect is expected in a neurodegenerative disorder such as APBD in which the already dead neurons cannot be affected by a post-onset treatment. This assumption was validated as for all the parameters improved by Compound 1—open field (FIG. 1I), weight (FIG. 1J) and overall extension reflex (FIG. 1K): Its ameliorating effect did not take place when it was administered after disease onset at the age 6 months. Notably, extension reflex, the parameter most affected by Compound 1, was also the only parameter improved by the compound from the advanced stage of the disease at the age of 9 months (FIG. 1K). The overall beneficial effect of Compound 1 can be best appreciated by animal photographs which illustrate that treated animals are less kyphotic and better kempt (FIGS. 1L-1M).

### EXAMPLE 2

### Compound 1 Reduces Histopathological Accumulation of Polyglucosans and Glycogen in Accordance With Its Biodistribution

As Compound 1 has significantly improved motor and survival parameters, the inventors set out to investigate its histopathological effects. This information is important for determining whether the expected mode of action of Compound 1 discovered ex vivo -reduction of polyglucosan levels in fibroblasts — also takes place in vivo and if so in which tissues. Brain, heart, muscle, nerve fascicles (peripheral nerves), and liver tissues from Compound 1 and vehicle treated animals were collected following animal sacrifice at the age of 9.5 months. The same tissues from wild type mice were used as controls. Following diastase treatment to digest non-polyglucosan glycogen, leaving behind polyglucosan, sections were stained for polyglucosan with periodic acid-Schiff s (PAS) reagent, counterstained with hematoxylin and analyzed by light microscopy. The results (FIG. 2A) show a significant reduction in polyglucosan levels in brain, liver, heart, and peripheral nerve, with no apparent effect on muscle polyglucosans. Total glycogen levels, determined biochemically, were also correspondingly affected (FIG. 2B). These results could possibly explain the improvement observed in motor parameters and in animal thriving (FIGS. 1A-1M).

Pharmacokinetic analysis is instrumental for explaining the effects of Compound 1 in-situ regardless of its innate capacity to modify polyglucosans in isolated cells. The reason for that is that timing of arrival, distribution and stability in the tissue are key determinants of the in-situ activity of any pharmacological agent. To determine the distribution and kinetic parameters of Compound 1 in different tissues, the inventors have treated Gbe^{ys/ys} mice with 250 mg/kg Compound 1 via subcutaneous injection, as done in the efficacy experiments. Mice were then sacrificed 0, 30-, 60-, 90-, and 210-min post administration and 100 μL serum as well as brain, kidney, hind limb skeletal muscle, heart, liver, and spleen tissues were collected, homogenized, extracted and their Compound 1 levels were analyzed by liquid chromatography tandem mass spectrometry (LC-MS/MS). The results are shown in FIG. 2C. The differential effects of Compound 1 on glycogen and polyglucosan content in the different tissues match its differential distribution and dwell time in each respective tissue. The highest extent of polyglucosan/glycogen reduction was observed in the liver matching the highest dwell time/persistence of Compound 1 observed in the organ (estimated half-life of more than 3 h). The heart and brain demonstrate intermediate levels of Compound 1. However, those levels persist up until 60 minutes post injection, which might account for the Compound 1-mediated reduction in polyglucosan, and glycogen content observed in these tissues. The muscle, on the other hand, demonstrates only negligible accumulation of Compound 1, in agreement with lack of effect of the compound on muscle glycogen and polyglucosan content. Based on the sampling times used, time to C. was 30 min for all the tissues studied indicating similar rate of absorption to all these tissues. The highest C. is observed in liver and kidney matching their well-established rapid perfusion. Expectedly, the lowest C. was observed in the skeletal quadriceps muscle, which is known to be a poorly perfused organ.

### EXAMPLE 3

### Compound 1 Enhances Carbohydrate Metabolism and Improves Metabolic Panel In Vivo

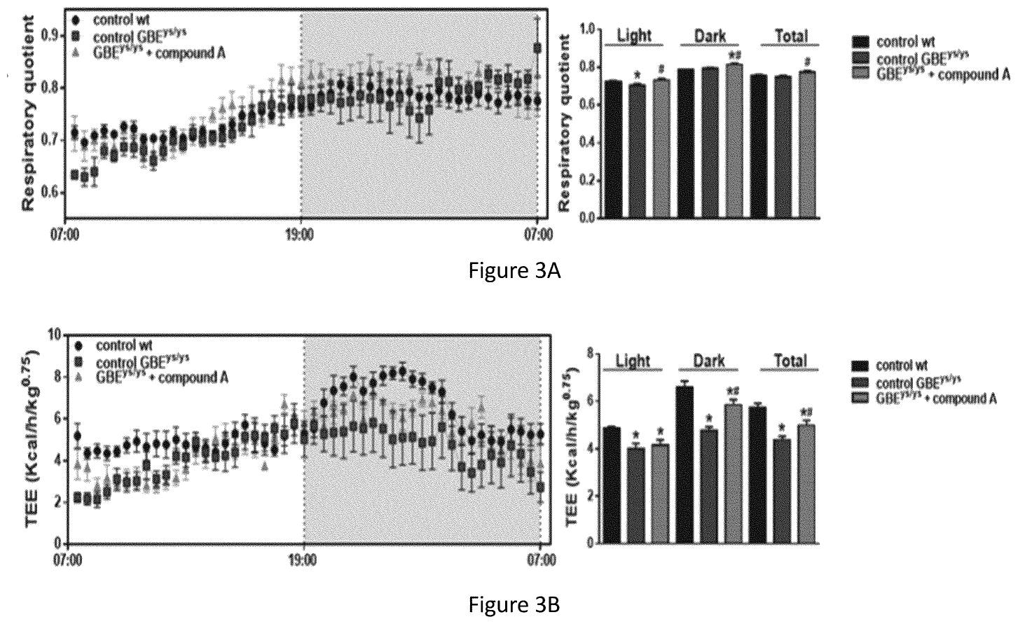

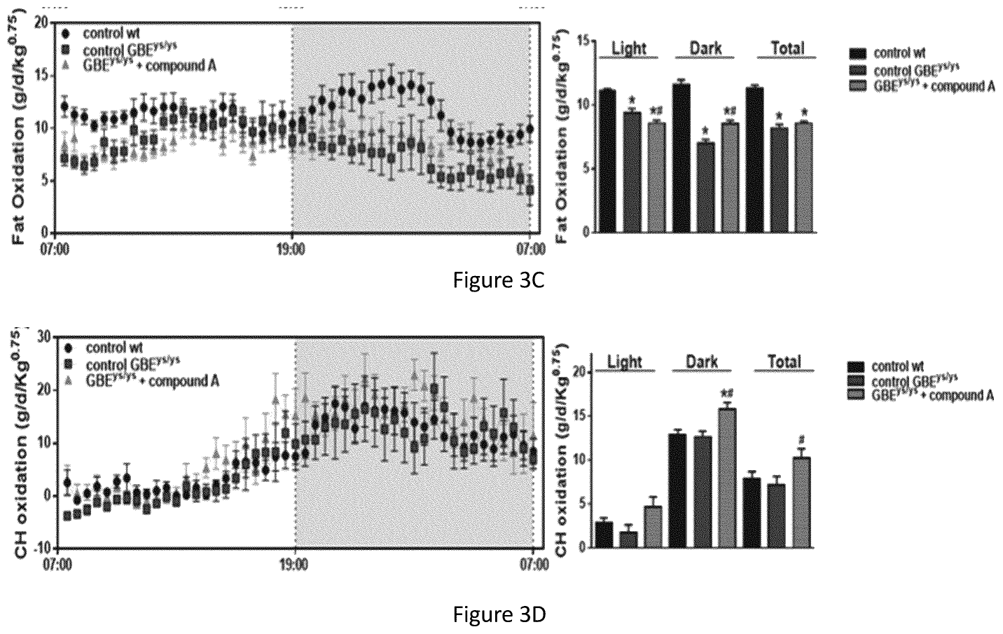

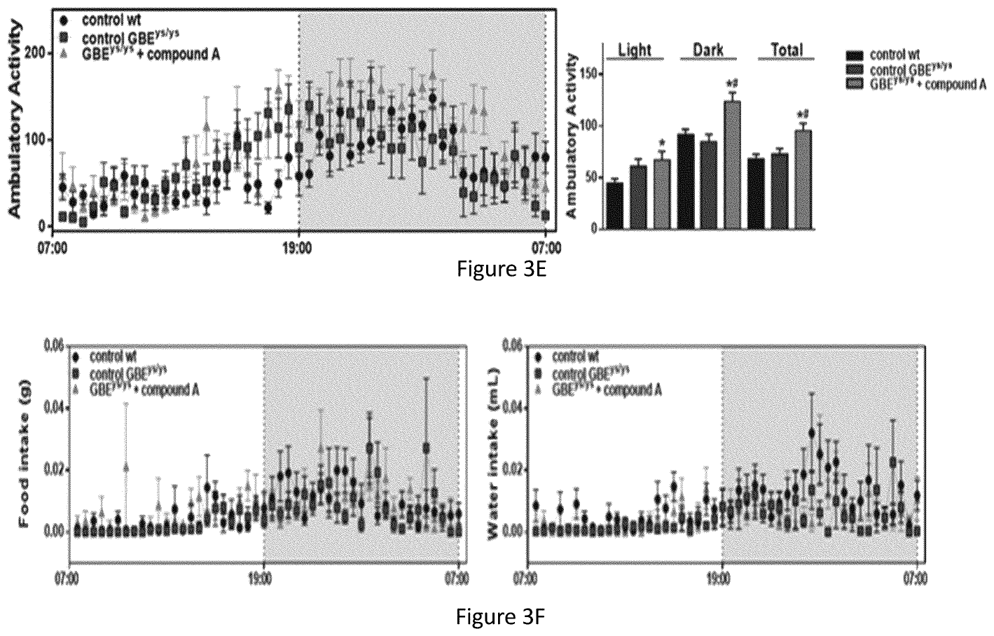

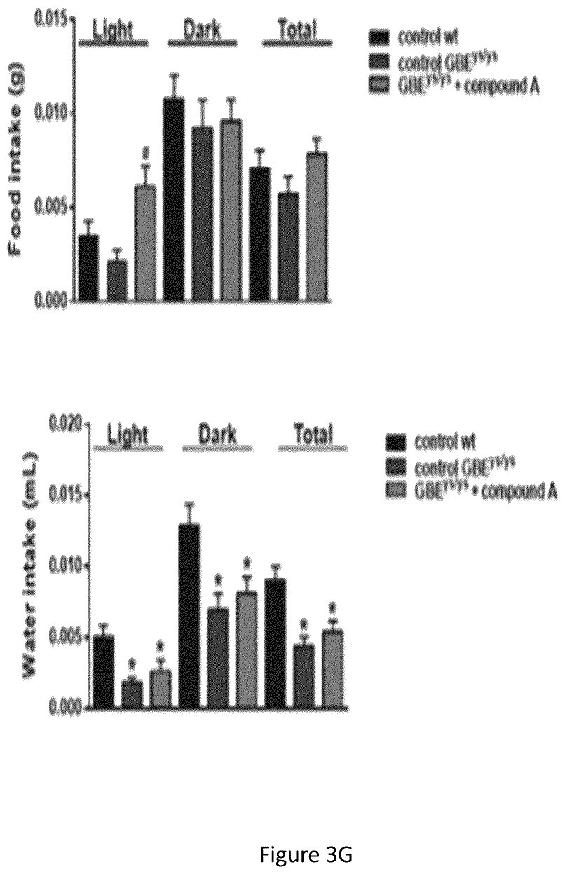

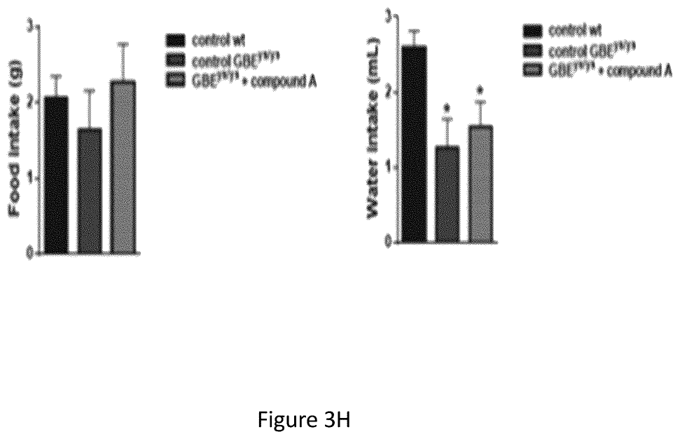

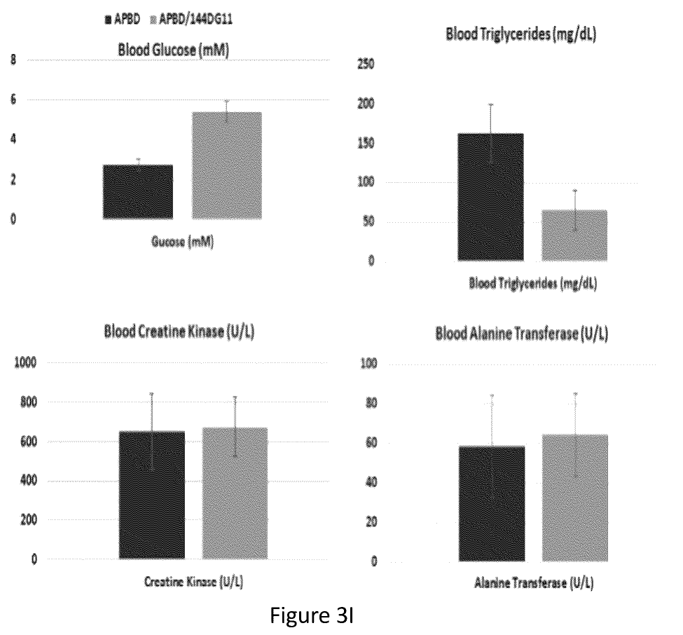

The effect of Compound 1 on various metabolic parameters was determined in vivo using metabolic cages. Fuel preference at the whole animal level is determined by the respiratory quotient (RQ, the ratio of CO₂ produced to O₂ consumed). Lower RQ indicates higher fat burn, while higher RQ indicates higher carbohydrate burn. As the results (FIG. 3A) show, Compound 1 has increased RQ to even higher levels than those of the wild type (wt) animals. The parallel increases, induced by Compound 1, in total energy expenditure (FIG. 3B) and carbohydrate burning at the expense of fat burning (FIGS. 3C and 3D) suggest that Compound 1 stimulates glycogen mobilization, which is a therapeutic advantage since Gbe^{ys/ys} mice store glycogen as insoluble and pathogenic polyglucosan. Stimulation of ambulatory activity (FIG. 3E) and of meal size and water intake (FIGS. 3F-3H) are in line with this observation of stimulation of carbohydrate catabolism in affected animals by Compound 1. Moreover, put together, the increased fuel burning, and food intake indicate that Compound 1 can improve metabolic efficiency in the affected animals.

The inventors further tested whether Compound 1 is able to correct the hypoglycemia and hyperlipidemia observed in Gbe^{ys/ys} mice. Such an effect is expected from an agent capable of inducing the catabolism of liver glycogen with an ensuing rise in blood glucose. The blood biochemistry test results of 9.5 months old Gbe^{ys/ys} mice demonstrate that upon treatment with Compound 1, the characteristic hypoglycemia and hyperlipidemia of the mice were corrected to control levels (FIG. 31). Muscle (creatine kinase) and liver (alanine transferase) functions were not affected by this treatment (FIG. 3I).

### EXAMPLE 4

### Compound 1 Enhances Catabolism in Glycogen Overloaded APBD Patient Cells

The RQ shift towards carbohydrate catabolism observed in vivo prompted the inventors to investigate whether carbohydrate catabolism is also up-modulated intracellularly. To that end, and especially since glycogen levels are highly versatile among fibroblasts derived from different APBD patients (FIG. 4A), the inventors first aimed at inducing a physiological glycogen overload, or glycogen burden condition, equivalent to the one found in tissues. The inventors found that glycogen burden can be produced by 48 h glucose starvation followed by replenishment of the sugar for 24 h, which possibly induces accelerated glucose uptake with ensuing glycogen synthesis. This starvation/replenishment condition indeed increased intracellular glycogen levels, as demonstrated by PAS staining (FIG. 4B). Furthermore, a multiparametric high-content imaging-based phenotyping analysis has revealed that under glycogen burden conditions, cell area, nuclear intensity and, importantly, mitochondrial mass features (see boxes in FIG. 4C) deviate from healthy control (HC) more than glucose starved-only cells do. Therefore, the inventors selected this glycogen burden condition to analyze catabolism at a cell level using an ATP Rate Assay (Agilent's Seahorse ATP Rate Assay). The results

(FIG. 4D) show that at the cell level, 144DG11 A has increased not only overall ATP production, but also the relative contribution of glycolytic ATP production at the expense of mitochondrial (OxPhos) ATP production. This phenomenon was observed in both HC and APBD patient skin fibroblasts. Acute on assay supplementation of 144DG11 was more effective at augmenting the glycolytic contribution to ATP production than 48 h pretreatment with the compound. These results suggest that glucose derived from the 144DG11-mediated enhanced carbohydrate catabolism is exploitable for ATP production.

### EXAMPLE 5

### Compound 1 Binds to the Lysosomal Membrane Protein LAMP1

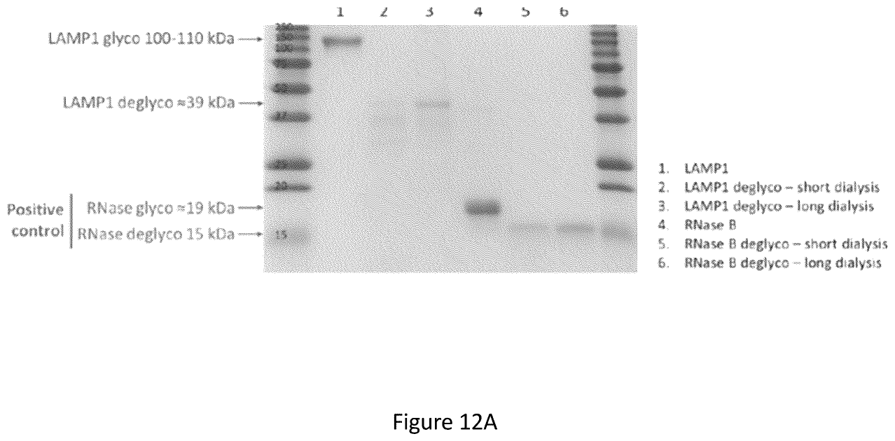

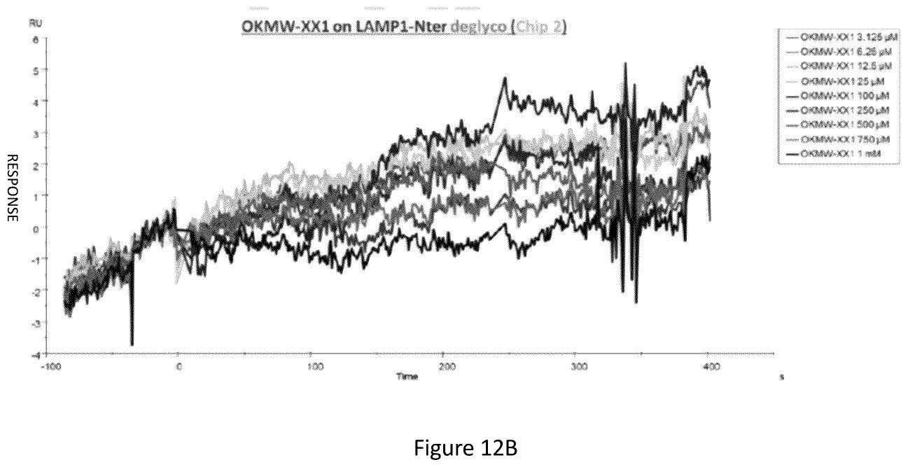

The inventors have investigated the mechanism of action of 144DG11. To that end, the inventors first decided to determine its molecular target. Nematic protein organization technique (NPOT, Inoviem, Ltd.) was applied to homogenates of APBD patient fibroblasts. The NPOT analysis has discovered protein hetero assemblies uniquely generated around 144DG11 only when it was added to the cell homogenates (FIG. 5A). The next step in this analysis has identified the interactome of protein targets interacting with 144DG11 in APBD patients' fibroblasts. Interestingly, as revealed by Inoviem's gene ontology analysis based on several bioinformatic tools, proteins in the hetero assembly interacting with 144DG11 in APBD patient fibroblasts are autophagic, or lysosomal proteins (FIG. 5B). Moreover, the inventors have tested by cellular thermal shift assay the specific interaction of 144DG11 with 6 of the 8 targets discovered by NPOT. The results (FIG. 5C) suggest that LAMP1, and not other protein targets, directly interacts with 144DG11. This finding relates to a novel pathogenic hypothesis connecting cellular glycogen overload with glycogen trafficking to lysosomes via Starch Binding Domain containing Protein 1. To validate 144DG11's interaction with LAMP1, the inventors have used surface plasmon resonance (SPR) technology. The SPR data (FIG. 5D) show a specific and dose-dependent binding of 144DG11 to the luminal portion of LAMP1 only at the lysosomal pH 4.5-5 and not at the cytoplasmic pH 7, with some binding starting at the intermediate pH 6. Put together, these results constitute strong and acceptable evidence that the specific target of 144DG11 is the type 1 lysosomal protein LAMP1, widely used as a lysosomal marker and a known regulator of lysosomal function. However, the apparent K_{D} of this binding is relatively high (6.3 mM), which is probably explained by the slow kₒₙ (rate of association in FIG. 5D, pH 4.5). The inventors hypothesized that this slow rate of association could be explained by inhibited diffusion of 144DG11 due to the bulky oligosaccharides at the glycosylation sites. Therefore, the inventors have repeated the SPR experiments with a chemically deglycosylated luminal LAMP1 domain. However, deglycosylated LAMP1 did not bind 144DG11, possibly due to profound structural changes induced by the deglycosylation (FIGS. 12A-12B), and therefore the inventors were unable to test whether oligosaccharide steric hindrance affects the binding kinetics of 144DG11 to LAMP 1. The inventors have further investigated 144DG11 binding to LAMP1 by structure-based computational docking. In the search for a putative binding site for Compound 1 in LAMP1, the inventors have analyzed the N- and C-terminal subdomains of its luminal domain (residues A29-R195 and S217-D378, respectively), which have a similar topology. These domains were modeled and computationally docked against decoys to Compound 1 at the intralysosomal pH 5 based on the known crystal structure of mouse LAMP1 C-terminal domain (PDB ID 5gv0). FIG. 5E shows the Compound 1 LAMP1 binding pocket (residues F50-D55, N62, L67, F118, Y120-L122, T125, L127-S133, N164-V166) predicted by three different algorithms: SiteMap, FtSite and fPocket. Prediction of the same binding site by three different programs is very rare and thus strongly suggests that Compound 1 binds to the specified site at the N-terminal of LAMP-1. As can be seen in FIG. 5E, Asn-linked oligosaccharides face away from the predicted Compound 1 binding site and are therefore not expected to directly interfere with its binding. However, they might still affect Compound 1 diffusion.

### EXAMPLE 6

### Compound 1 Enhances LAMP1 Knockdown-Induced Autolysosomal Degradation and Catabolism of Glycogen

Compound 1 has increased autophagic flux in APBD primary fibroblasts. This is demonstrated by an increased sensitivity to lysosomal inhibitors in the presence of Compound 1. As can be seen in FIG. 6A, lysosomal inhibitors increase the LC3ii/LC3i ratio (autophagic halt) more in Compound 1 treated than in untreated cells. Increase in autophagic flux by Compound 1 is also illustrated by lowering the level of the autophagy substrate p62 (FIG. 6A). Moreover, transmission electron microscope analysis of liver sections of the APBD modeling Gbe^{ys/ys} mice demonstrates a decrease in lysosomal glycogen following treatment with Compound 1 (FIG. 6B).

To determine the functional importance of the interaction between Compound 1 and LAMP1, the inventors knocked down the latter using a lentiviral vector carrying GFP tagged shRNA against LAMP1. As LAMP1 knockdown (KD) becomes cytotoxic 24 h post expression (or 96 h post lentiviral infection), LAMP1-KD experiments in FIGS. 6C-6D were conducted under 24 h serum starvation condition, without glucose replenishment (FIGS. 4A-4D), to both induce autophagy and maintain cell viability. The inventors expected LAMP1-KD to neutralize the effect of Compound 1 allegedly mediated by its interaction with LAMP1. Surprisingly, however, supplementation of Compound 1 to LAMP1 knocked down cells enhanced the knockdown effect: Autophagic flux, enhanced by LAMP1-KD, was further enhanced by the LAMP1 interacting Compound 1 (FIG. 6C). The observation that Compound 1 enhances LAMP1-KD effect suggests that the interaction of Compound 1 with LAMP1 is inhibitory, as many other small molecule-protein interactions are. Furthermore, to test whether LAMP1-KD and Compound 1 enhanced autophagic flux by improving lysosomal function, the inventors quantified lysosomal acidification using the pH ratiometric dye Lysosensor™, which quantifies pH based on the yellow/blue emission ratio. The results show that both LAMP1-KD and Compound 1 treatment (in GFP and LAMP1-KD APBD cells) led to lysosomal acidification, but more so LAMP1-KD. The inventors show by flow cytometry (FIG. 6D, upper panel) the overall cellular acidification as an increase in 375 nm-excited yellow/blue emission, and by confocal microscopy that this acidification is associated with brighter yellow fluorescence in lysosomes (FIG. 6D, middle panel). Importantly, as demonstrated by PAS staining, LAMP knockdown reduced cellular glycogen levels, an effect which was slightly enhanced by Compound 1 in APBD fibroblasts transduced with both GFP control and shLAMP1-GFP lentiviruses (FIG. 6D, lower panel).

To test the effect of LAMP1-KD and Compound 1 on fuel utilization, the inventors again used the ATP Rate Assay (FIGS. 4A-4D) in LAMP1-KD and control APBD fibroblasts acutely or chronically treated with Compound 1. The results (FIG. 6E) show that starvation was more restrictive (lowered overall ATP production) in LAMP1-KD (LAMP1-KD-S UT v LAMP1-KD+S UT, p<0.0001) than in GFP-transduced controls (Control UT-S v Control UT+S, p<0.36). In LAMP1-KD cells, starvation also increased the relative contribution of respiration to ATP production (78% in LAMP1-KD-S UT v 48% in LAMP1-KD+S UT (orange bars)). These observations are in line with the higher ATP production efficiency of respiration as compared to glycolysis and possibly with higher ATP demand of LAMP-KD, as compared to control cells, as suggested by their higher overall ATP production rate in basal conditions (cf. LAMP1-KD+S UT with Control+S UT, p<0.01). The effect of Compound 1 on LAMP1-KD and control cells was in accordance with its selective increase of catabolic (ATP generating) autophagic flux in LAMP1-KD cells, as compared to control cells (FIG. 6C): In non-starved conditions, supplementation of Compound 1 significantly increased total and respiratory ATP production in LAMP1-KD cells (cf. LAMP1-KD+S UT with LAMP1-KD+S Chronic (p<0.03 for total, p<0.0008 for respiratory) and LAMP1-D+S Acute (p<0.01 for total and respiratory)), while in control cells it only slightly influenced ATP production, and even acutely decreased it (cf. Control UT+S with Control+S Chronic (p<0.1) and Control UT+S Acute (p<0.0008 for decrease)). Under starved conditions, control cells only increased respiratory ATP production in response to the transient effects of acutely supplemented Compound 1 (cf. Control UT-S with Control-S Acute, p<0.004). No significant effect of chronic supplementation of Compound 1 was observed in control cells (cf. Control UT-S to Control-S Chronic, p<0.3). In contrast, starved LAMP1-KD cells increased both respiratory and glycolytic ATP as a response to acute supplementation of Compound 1, possibly reflecting short-term diversion of glucose derived from glycogen degradation to glycolysis (cf. LAMP1-KD-S UT with LAMP1-KD-S Acute (p<0.0003 for glycoATP, p<0.003 for mitoATP). In response to chronically administered Compound 1, only respiratory ATP production increased (cf. LAMP1-KD-S UT with LAMP1-KD-S Chronic (p<0.15 for glycoATP, p<0.0002 for mitoATP) in LAMP-KD cells.

### EXAMPLE 7

### Compound 1 Restores Aberrant Mitochondrial and Lysosomal Features at the Cell Level

As the inventors showed that the mode of action of Compound 1 involves lysosomal catabolism which increases ATP production, the inventors decided to investigate whether the cellular features modulated by Compound 1 are relevant to its catabolic effects. As a first step, the inventors required a classification method, both wholistic and feature-specific, which would enable to quantify differences between APBD and HC cells and thus to estimate the restorative effect of Compound 1 on APBD cells. Using the InCell2200 high-content image analyzer, the inventors have conducted a thorough multi-parametric analysis of APBD and age and gender matched HC skin fibroblasts. This image-based phenotyping (IBP) campaign included 45 independent cellular parameters encompassing a wide cell-morphological spectrum. Analyzing skin fibroblasts from 17 APBD patients and 5 HC, the inventors have demonstrated that skin fibroblasts from APBD patients are phenotypically distinguishable from HC skin fibroblasts (FIG. 7A). Once IBP was established as an informative and sensitive classification tool, the inventors tested the effect of Compound 1 on the IBP signature: The analysis (FIG. 7B, upper panel), which was limited to 4 color channels and thus excluded a lysosomal marker, analyzed separately (FIG. 7C), shows that Compound 1 has mostly affected nuclear and mitochondrial membrane potential (TMRE) parameters, which were among the features most affected by the disease phenotype. As expected, this effect was more pronounced (higher -logP value) when Compound 1 treated APBD fibroblasts were compared to untreated HC fibroblasts (a comparison more relevant to the clinical settings). As demonstrated for other features (FIGS. 4D and 6C-6E), Compound 1 treatment on its own likely has similar effects on both affected and healthy cells and therefore likely brings the two phenotypes closer together in treated cells, partially masking the effect of this compound on APBD v HC. The lower panel in FIG. 7B indeed reveals that, for most features, Compound 1 had caused the same trend (increase or decrease) in both affected and healthy cells (note that APBD/Compound 1 (stippled bars) should be compared to APBD (blank bars), and HC/Compound 1 (black bars) should be compared to the horizontal line). Compound 1 has also reduced lysosomal size in APBD cells (FIG. 7C), which could be associated with its improvement of autophagic flux (FIG. 6) and lysosomal function, as observed in healthy as compared to lysosomal impaired cells. Moreover, Compound 1 has also hyperpolarized the mitochondrial membrane potential (MMP), depolarized by the diseased state in APBD (FIG. 7B), in accordance with possibly increased mitochondrial fueling by the enhanced autophagic catabolism.

To validate the imaging-based analysis of the cell features modulated by the diseased state and by Compound 1, the inventors analyzed the effect of the disease and the treatment on protein expression. As shown in FIG. 7D, under 48 h starvation 12.2% and 6.8% of the 2,898 proteins analyzed were respectively up and down modulated in APBD-patient as compared to HC cells. As an important control, GBE was indeed downmodulated in the APBD cells (FIG. 7D). When starvation was followed by glucose supplementation (glycogen burden, FIGS. 4A-4D), only 6% of the proteins were up-modulated and 5% down-modulated, possibly suggesting a more specific subset of proteins was required for managing the excess glycogen burden. For instance, autophagy proteins (Fycol, Rab12, Rab7A, PIP4K2B, SQSTM1, and SNAP29) were only up modulated in APBD cells following glycogen burden. The inventors then investigated the proteomic effect of Compound 1 in starved (48 h starvation) and glycogen overladen (48 h starvation/24 h Gluc) APBD cells, which respectively modified only 1.7% and 1.3% of all proteins. The apparently corrective effect of Compound 1 can be uncovered by proteins down-modulated or up-modulated by the APBD diseased state, which were inversely up-modulated or down-modulated by Compound 1 (FIG. 7E). The discovered proteins (49 up-modulated, 39 down-modulated, FIG. 7E) were analyzed by the DAVID functional annotation tool according to the Cellular Component category, which included the highest number of proteins. Proteins up modulated by Compound 1 belonged to 8 significant gene ontology (GO) terms, which included lysosomal, secretory pathways and oxidative phosphorylation proteins (FIG. 7F, left panel) in accordance with the cell features modulated by the compound (FIG. 7B).

Interestingly, proteins down-modulated by APBD and up-modulated (“corrected”) by Compound 1 were the lysosomal glycosylation enzymes Iduronidase and Phosphomannomutase2 under glycogen burden, whereas under starvation those were the nucleic acid binding proteins GRSF1 and HNRPCL1, apparently not directly associated with glycogen and lysosomal catabolism. The lipogenetic protein HSD17B12 was decreased by APBD and induced by Compound 1 under both conditions. Proteins downmodulated by Compound 1 belonged to 4 GO terms, which included secretory pathways and macromolecular complexes (FIG. 7F, right panel). Proteins increased by APBD and contrarily reduced by Compound 1 belonged to lysosomal sorting (VPS16) and carbohydrate biosynthesis (NANS) in starved cells and to transcription (RUBL1), signal transduction (STAM2) and pH regulation (SLC9A1) in glycogen overladen cells. Interestingly, pharmacological inhibition of the Na⁺/H⁺ antiporter SLC9A1 induces autophagic flux in cardiomyocytes as does its down-modulation in APBD fibroblasts by Compound 1 (FIG. 6). The protein downmodulated by Compound 1 in both starved and glycogen burden conditions is the retrograde traffic regulator VPS51 also implicated in lysosomal sorting. In summary, the APBD correcting effects of Compound 1 are at least partially related to lysosomal function whose modulation by the compound is well established by the inventors (FIGS. 5-6).

This work shows that the HTS-discovered hit Compound 1 can remedy APBD in in vivo and ex vivo models. Following Compound 1 treatment, the inventors observed improvements in motor, survival, and histological parameters (FIGS. 1-2). As APBD is caused by an indigestible carbohydrate, these improvements suggested that Compound 1 affected carbohydrate metabolization and thus encouraged the inventors to conduct in vivo metabolic studies (FIGS. 3A-3I). This is the first in vivo metabolic study in a GSD animal model. Since APBD mice store glycogen as insoluble polyglucosan, the inventors used metabolic cages to test whether Compound 1 can influence the capacity of these animals to use alternative fuels (fat) instead of mobilizing glycogen. However, the increased RQ induced by Compound 1 suggested that instead of using fat, treated animals actually increased carbohydrate burn, or that Compound 1 increased carbohydrate catabolism. This conclusion was supported by the Compound 1-induced increases in total energy expenditure, ambulatory activity, meal size and water intake - all in line with catabolic stimulation. Since Gbe^{ys/ys} mice and APBD patients store glycogen as insoluble and pathogenic polyglucosan, its catabolism constitutes a therapeutic advantage. Glycogen catabolism is also a preferred therapeutic strategy for the following reason: In theory, therapeutic approaches to APBD should target either PG formation, or degradation of preformed PG or glycogen. PG formation depends on the balance between GYS and GBE activity — the higher the GYS/GBE activity ratio, the more elongated and less branched soluble glycogen would form, which would preferentially form PG, as compared to shorter chains. Degradation of pre-existing PG and glycogen (PG precursor), on the other hand, as done by Compound 1, is a more direct target and is expected to be more efficacious than inhibition of de novo PG formation, as done by the GYS inhibitor guaiacol, which spares pre-made detrimental PG. Indeed, in a study in LD-modeling mice, it was shown that conditional GYS knockdown after disease onset is unable to clear pre-existing and detrimental Lafora PG bodies.

A key challenge in drug discovery is the determination of relevant targets and mechanism of action of drug candidates. To that end, the inventors have applied here Inoviem's NPOT® protein target identification approach. This technique, recognized as a leading tool for identifying protein targets of small molecules, and which identified several therapeutically relevant targets, identifies compound-target interactions within the natural physiological environment of cells. This means that the entity identified is not the target per se, as in other technologies, but the primary target with its signaling pathway, or functional quaternary network. Determination of the cellular pathway modulated by the test compound, as done for Compound 1, is important for putative formulation of other drugs to the same pathway, which can significantly upgrade therapeutic efficacy in due course in the clinic. Moreover, NPOT® can also confirm the specificity of target binding by filtering out promiscuous binders and excluding binding to negative controls (in this case, negative compounds in the HTS) and to endogenous ligands (FIG. 5A). Nevertheless, while by these criteria Compound 1 binding to LAMP1, and through it to its functional quaternary network (FIG. 5B), was specific and manifested dose response and lysosomal pH dependence in the SPR validation (FIG. 5D), its apparent LAMP 1 binding KD was relatively high (6.3 mM), which seemingly could be an impediment towards its clinical application. This issue can be coped with as follows: 1. The pharmacologically relevant finding is that Compound 1 specifically interacted with a lysosomal-autophagosomal interactome (FIG. 5B) and that it was not toxic (FIGS. 8-11, Table 2).

This finding rules out non-specific interaction with putative off-targets, which is the main concern in low affinity (high KD) ligands. A conventional approach to improve the affinity of low-affinity pharmaceutical candidates is based on medicinal chemistry. In GSDs, such an approach was used for increasing the affinity of GYS inhibitors. However, as opposed to GYSs, whose reduction is relatively tolerable, the LAMP proteins belong to the house keeping autolysosomal machinery (FIG. 5B), whose inhibition can compromise perinatal viability, as does, for instance, LAMP1-KD without a compensatory rise in LAMP2. Therefore, a high affinity LAMP1 inhibitor might be toxic, as was LAMP1-KD to APBD fibroblasts (FIG. 6), and the low affinity of the LAMP1 inhibitor the inventors discovered, Compound 1, may actually constitute a clinical advantage by mitigating the repression of a household function. Moreover, the computational analysis indicates that the Compound 1 binding pocket in LAMP1 (FIG. 5E) is highly druggable, i.e., medicinal chemistry analysis is expected to discover various alternatives to Compound 1 which could improve its effect.

The discovery of the LAMP1 containing hetero assembly (FIG. 5B) as a functional network target, rather than a single protein, opens a therapeutic modality based on autophagy modulation, which actually expands the therapeutic target landscape. Autolysosomal network was discovered not only in FIG. 5B, but also by the inventors' multi-feature imaging analysis, in conjunction with bioenergetic parameters, possibly modified by autophagy-associated changes in fuel availability (FIGS. 7B and 7C). Additional support for the relevance of this pathway as a Compound 1 target come from the proteomics data (FIGS. 7D-7F) and from the actual boost of autophagic flux by Compound 1 in cells (FIG. 6).

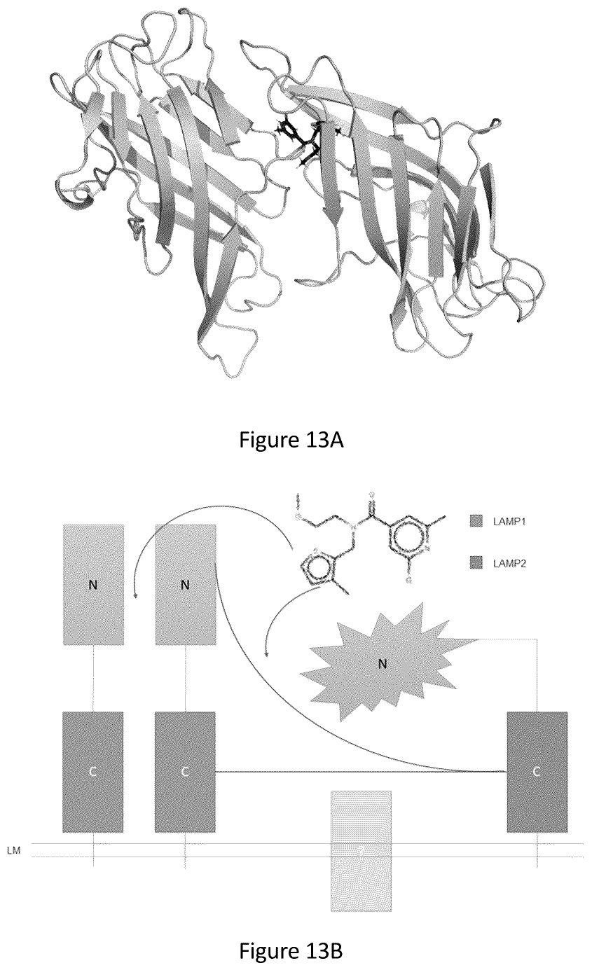

Mechanistically, LAMP1 is a type I lysosomal membrane protein which, together with LAMP2, plays a pivotal role in lysosome integrity and function. Consequently, LAMP1, but more so LAMP2, are also important for lysosomal involvement in the autophagy process. Therefore, LAMP1 knockdown is often associated with decreased autophagy. However, in agreement with the present results, other works show that LAMP1-KD actually increased autophagic function, which was also shown for another transmembrane lysosomal protein TMEM192. These apparent disparities probably depend on cell type, assay conditions, and even the definition of autophagy, as autophagic flux is not always defined by susceptibility to lysosomal inhibitors. To predict the molecular mechanism of action of Compound 1 on LAMP1, the inventors used computational chemistry. The computational results predict that the Compound 1 binding site is located at the LAMPl:LAMP1 interaction interface (FIG. 13A) (located at the N-terminal domain) and suggest that the compound inhibits inter-LAMP1 interaction. According to experimental data, truncation of the N-terminal domain of LAMP1 impairs LAMP 1/LAMP1 and LAMP 1/LAMP2 assembly, while truncation of the more mobile LAMP2 N-terminal domain leads to the opposite effect (FIG. 13B). Therefore, the inventors may assume that LAMP1 N-terminal domain promotes LAMP1/LAMP1 and LAMP1/LAMP2 interactions and that inhibition of LAMP1/LAMP1 or LAMP1/LAMP2 interactions at the N-terminal domain, by Compound 1, would lower LAMP1 effective lysosomal membrane density. Thus, Compound 1 treatment can be hypothesized to be equivalent to LAMP1-KD, which might explain its enhancement of the LAMP1-KD effect. The slight increase (1.2-fold) in LAMP1 levels induced by Compound 1 probably reflects binding-mediated stabilization (FIG. 5C) and presumably does not significantly counteract Compound 1-mediated reduction in membrane density. The inventors hypothesize that the Compound 1-mediated decrease in LAMP1 membrane density increases glycophagy by the documented increase in LAMP2 in lysosomal membranes upon LAMP1-KD. LAMP2 was observed to enhance autophagosome-lysosome fusion (and thus autophagic flux) by interaction with the autophagosomal peripheral protein GORASP2. Alternatively, spacing of the lysosomal membrane by LAMP1-KD/Compound 1 may enable glycogen import to the lysosome (and consequent degradation) by the STBD1 protein. Importantly, lysosomal glycogen degradation takes place in parallel with its cytoplasmic degradation, and, specifically, in a GSDIV mouse model, which also models APBD in mice, overexpression of the lysosomal glycogenase a-glucosidase corrected pathology.

In summary, this work demonstrates Compound 1 as a novel catabolic compound capable of degrading PG and over-accumulated glycogen by activating the autophagic pathway. This study lays the groundwork for clinical use of Compound 1 in treating APBD patients who currently have no therapeutic alternative. Moreover, it positions Compound 1 as a lead compound for treating other GSDs through safe reduction of glycogen surcharge.

### EXAMPLE 8

### Therapeutic Attributes of the 144DG11 Compound

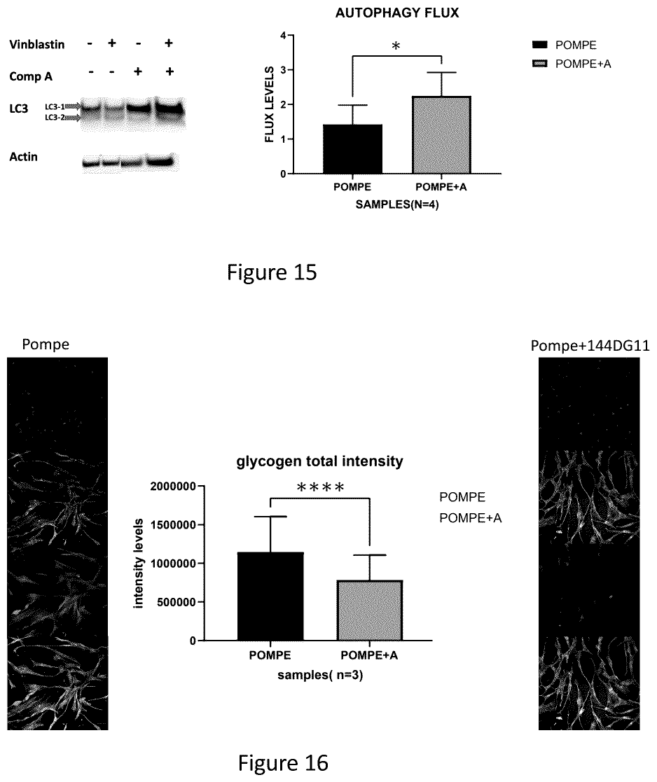

144DG11 can activate autophagy in the lysosomal storage disease (LSD) Pompe disease (PD) in which autophagy is perturbed (FIG. 15). The data show that in fibroblasts derived from PD patients, the ratio of the lipidated autophagic marker LC3 (LC3II) to non-lipidated LC3 (LC3I) LC3 was increased by the autolysosomal inhibitor vinblastine. This ratio serves as the most accepted marker for autophagy and autophagic flux. Sensitivity to vinblastine (i.e., increase in the LC3II/LC3I ratio, demonstrating accumulation of non-degraded autophagic substrate) was increased by treating serum starved PD patient-derived fibroblasts with 50 μM 144DG11 for 24 h. These observations indicate that, as in the GSD APBD, 144DG11 can activate autophagy also in the typical LSD PD in which autophagy is disturbed. This strongly suggests that 144DG11 has a therapeutic potential also for treating LSDs in which perturbation of autophagy is a leading pathogenic factor.

144DG11 (24 h, 50 μM) can reduce glycogen in PD patient-derived fibroblasts, as was also demonstrated in APBD patient-derived fibroblasts (FIG. 16).

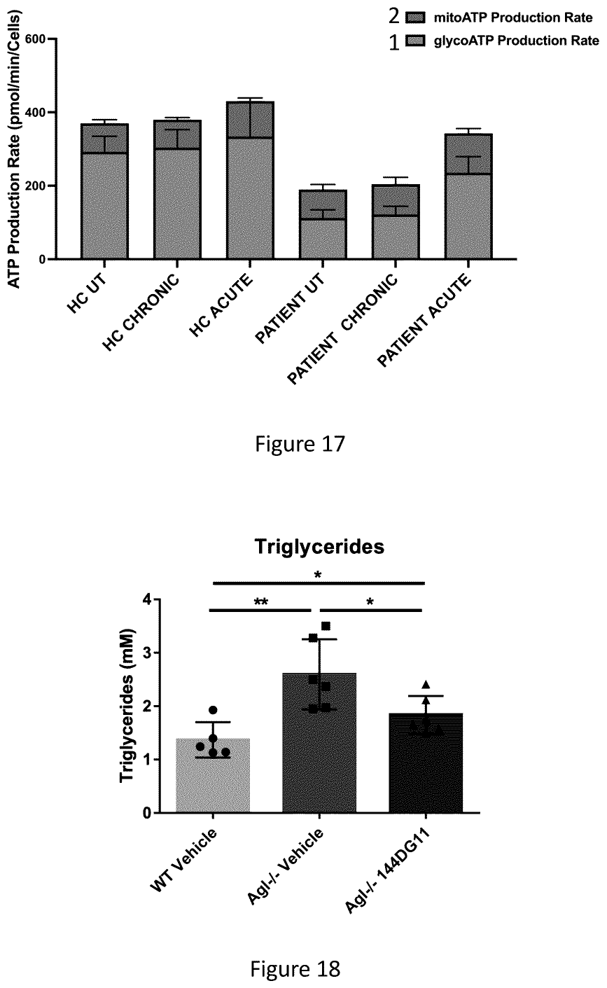

The results (FIG. 17) show that 144DG11 increased overall ATP production as well as the relative contribution of glycolytic ATP production at the expense of mitochondrial (OxPhos) ATP production. This phenomenon was observed selectively in PD and not in healthy control (HC) primary skin fibroblasts. Assay supplementation of 144DG11 was more effective at augmenting the glycolytic contribution to ATP production than 24 h pre-treatment with the compound, whose effect on ATP production was not significant, possibly due to cellular adaptation. These results suggest that glucose derived from the 144DG11-mediated enhanced autophagic catabolism is exploitable for ATP production. These observations are in agreement with the observations made in APBD fibroblasts and thus demonstrate 144DG11 as a general catabolic enhancer with wide therapeutic capacities in storage disorders in general.

The results in FIG. 17 suggesting that the glucose derived from the enhanced carbohydrate catabolism is exploitable for ATP production, support future development of 144DG11 as an effective anti-obesity medication. The inventors expect 144DG11 to be more effective in western diet (high fat/high carb)-induced obesity. The observations in GSD4 (Kakhlon et al., (2021)) and GSD3 (FIG. 18) that 144DG11 can decrease the level of plasma triglycerides strongly suggest that 144DG11 can be developed into an effective anti-obesity therapy.

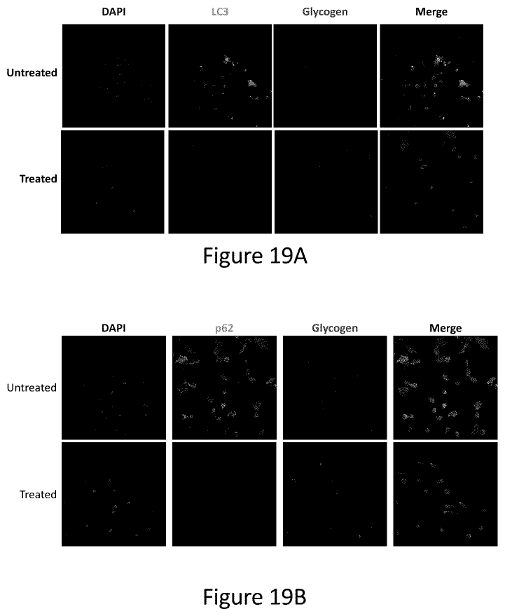

As shown by the 144DG11-mediated decrease in total LC3 and p62, the compound induced autophagy in brain microglia derived from Alzheimer's Disease (AD) modeling mice (FIG. 19). This observation is important as it demonstrates a therapeutic potential of 144DG11 for treating AD. Being the most pro-inflammatory tissue in the brain, microglia are currently at the epicenter of innovative therapeutic research for AD. Moreover, as neuroinflammation is now accepted as the main pathogenic factor in AD and as activation of microglia autophagy and mitophagy is a leading therapeutic strategy (see, for instance, Eshraghi et al., (2021)), 144DG11 holds promise as a potential AD therapeutic.

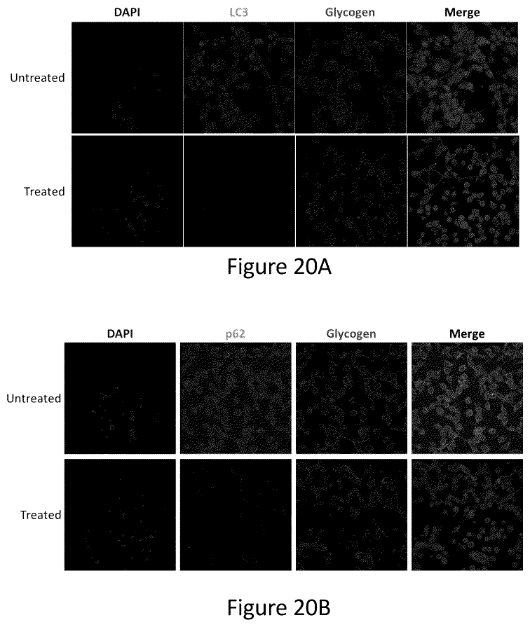

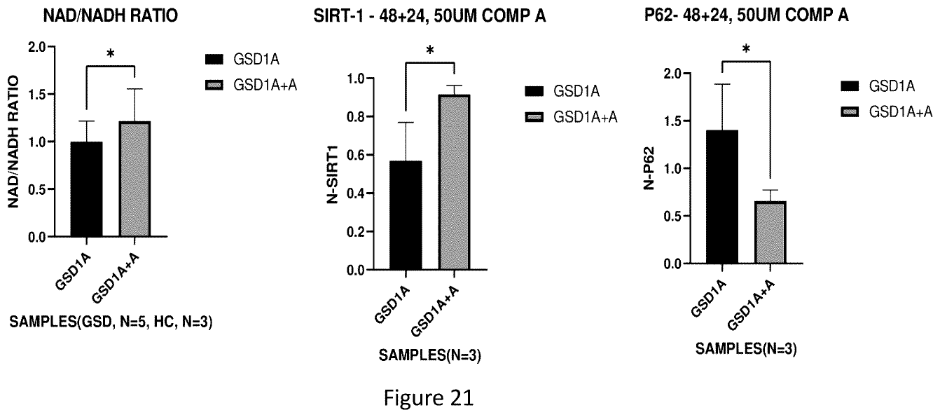

144DG11 also induced autophagy in primary human non-small-cell lung cancer (NSCLC) cells (FIG. 20). Induction of autophagy has a demonstrated therapeutic value in NSCLC (for example, see Wang et al., (2021)). Notably, 144DG11 did not lower glycogen levels in both microglia and NSCLC cells, suggesting that glycogen is not degraded by an autolysosomal pathway, which is modifiable by 144DG11, in these cells. This lack of effect on glycogen also suggests that glycogen accumulation may not be pathogenic in these cells. However, autophagic clearance of noxious inclusions by 144DG11 is probably beneficial in many different disease states as demonstrated here. NAD+ and NADH are key precursors for the electron transfer chain, TCA cycle, glycolysis, amino acid synthesis, fatty acid synthesis, and nucleotide synthesis. The NAD+/NADH ratio reports the extent of overall catabolism and the balance between glycolysis and OxPhos. The increase in the NAD+/NADH ratio means acceleration of electron flow in the mitochondrial electron transport chain (note, not mitochondrial ATP production) and of glycolysis to better manage metabolic demands. Furthermore, Sirt1 induction, often associated with increased NAD+/NADH ratio, is a well-documented and innovative anti-aging, calorie restriction-mimic and anti-cancer therapeutic strategy (see for example Hyun et al., (2020)). Thus, the results in Gsd1a cells (FIG. 21), showing induction of the NAD+/NADH ratio, as well as Sirt1, indicate that 144DG11 is a promising therapy for a plethora of different metabolic disorders, aging-related complications, and cancer. In addition, 144DG11 downmodulated p62 indicating an increase in autophagic flux in Gsd1a cells as demonstrated in GSD4 and PD cells.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent, or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

1-21. (canceled)

22. A method for treating a lysosomal storage associated disease selected from the group consisting of a mucopolysaccharidosis (MPS), Gaucher disease, Fabry disease, Tay-Sachs disease, aspartylglucosaminuria, GM1-gangliosidosis, Krabbe disease, metachromatic leukodystrophy, Sandhoff disease, a mucolipidosis, Niemann-Pick disease, free sialic acid storage disorder, multiple sulfatase deficiency (MSD), a sphingolipidosis, Farber disease, GM2 gangliosidosis, lysosomal acid lipase deficiency, a neuronal ceroid lipofuscinosis, Wolman disease, cholesteryl ester storage disease, galactosialidosis, alpha-mannosidosis, beta-mannosidosis, fucosidosis, Salla disease, Schindler disease, cystinosis and pycnodysostosis in a subject in need thereof, the method comprising administering to the subject a pharmaceutical composition comprising a compound, pharmaceutically acceptable salt, isomer, or tautomer thereof, wherein said compound is represented by Formula I: 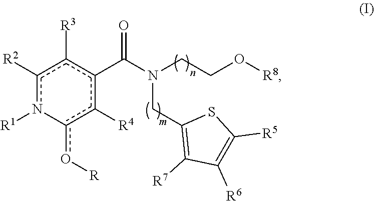
wherein: [symbol]
represents a single or a double bond;
n and m each independently represents an integer in a range from 1 to 3;
R and R¹ each independently represents hydrogen, or is absent; and
R³, R⁴, R⁵, R⁶, R⁷ and R⁸ each independently represents hydrogen, or is selected from the group comprising alkyl, cycloalkyl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, substituted or non-substituted.

23. The method of claim 22, wherein the 1VIPS is selected from the group consisting of MPS Type I-H (Hurler syndrome), MPS Type I-S (Scheie syndrome), MPS Type I-H S (Hurler-Scheie syndrome), 1VIPS Type II (Hunter syndrome), MPS Type III (Sanfilippo syndrome), MPS Type IV (Morquio syndrome), MPS Type VI (Maroteaux-Lamy syndrome), MPS Type VII (Sly syndrome), and MPS Type IX (hyaluronidase deficiency).

24. The method of claim 22, wherein n and m is 1.

25. The method of claim 22, wherein R² , R⁷ and R⁸ represent a methyl.

26. The method of claim 22, wherein said compound is selected from: 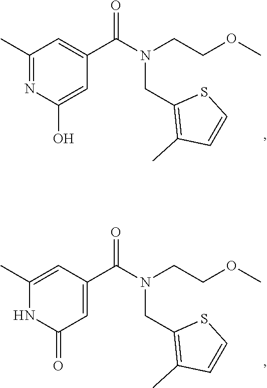
or both.

27. A method for treating a psychiatric disorder associated with autophagy misregulation in a subject in need thereof, the method comprising administering to the subject a pharmaceutical composition comprising a compound, pharmaceutically acceptable salt, isomer, or tautomer thereof, wherein said compound is represented by Formula I: 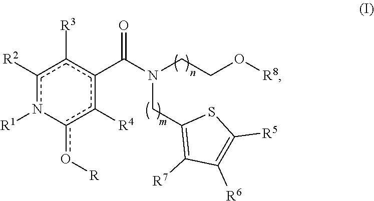
wherein: [symbol]
represents a single or a double bond;
n and m each independently represents an integer in a range from 1 to 3;
R and R¹ each independently represents hydrogen, or is absent; and
R³, R⁴, R⁵, R⁶, R⁷ and R⁸ each independently represents hydrogen, or is selected from the group comprising alkyl, cycloalkyl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, substituted or non-substituted.

28. The method of claim 27, wherein the psychiatric disorder is selected from schizophrenia and bipolar disorder.

29. The method of claim 27, wherein n and m is 1.

30. The method of claim 27, wherein R², R⁷ and R⁸ represent a methyl.

31. The method of claim 27, wherein said compound is selected from: 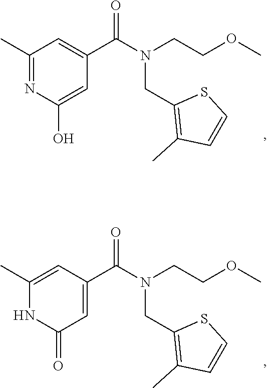
or both.

32. A method for treating lung cancer in a subject in need thereof, the method comprising administering to the subject a pharmaceutical composition comprising a compound, pharmaceutically acceptable salt, isomer, or tautomer thereof, wherein said compound is represented by Formula I: 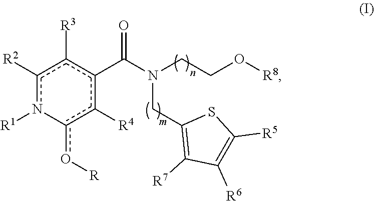
wherein: [symbol]
represents a single or a double bond;
n and m each independently represents an integer in a range from 1 to 3;
R and R¹ each independently represents hydrogen, or is absent; and
R³, R⁴, R⁵, R⁶, R⁷ and R⁸ each independently represents hydrogen, or is selected from the group comprising alkyl, cycloalkyl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, substituted or non-substituted.

33. The method of claim 32, wherein the lung cancer is non-small-cell lung cancer (NSCLC).

34. The method of claim 32, wherein n and m is 1.

35. The method of claim 32, wherein R², R⁷ and R⁸ represent a methyl.

36. The method of claim 32, wherein said compound is selected from: 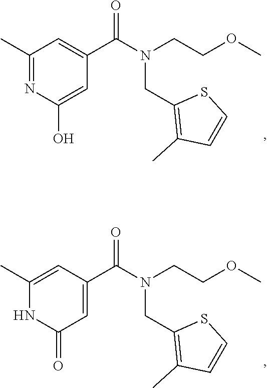
or both.

37. A method for treating an ocular disorder associated with autophagy misregulation in a subject in need thereof, the method comprising administering to the subject a pharmaceutical composition comprising a compound, pharmaceutically acceptable salt, isomer, or tautomer thereof, wherein said compound is represented by Formula I: 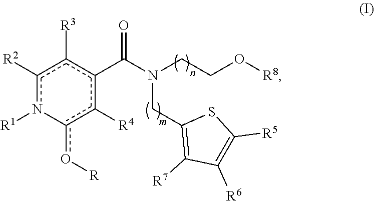
wherein: [symbol]
represents a single or a double bond;
n and m each independently represents an integer in a range from 1 to 3;
R and R¹ each independently represents hydrogen, or is absent; and
R³, R⁴, R⁵, R⁶, R⁷ and R⁸ each independently represents hydrogen, or is selected from the group comprising alkyl, cycloalkyl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, substituted or non-substituted

38. The method of claim 37, wherein n and m is 1.

39. The method claim 37, wherein R², R⁷ and R⁸ represent a methyl.

40. The method of claim 37, wherein said compound is selected from: 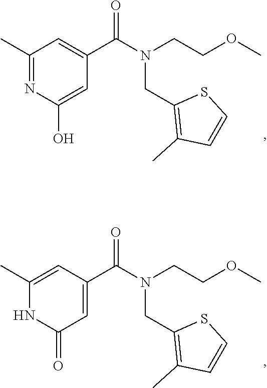
or both.


SEQUENCE LISTING (4 sequences)

SEQ ID NO: 1 - protein, 64 aa, Artificial Sequence
  Synthetic
FSVNYDTKSGPKNMTFDLPSDATVVLNRSSCGKENTSDPSLVIAFGRGHTLTLNFTRNATRYSV

SEQ ID NO: 2 - protein, 6 aa, Artificial Sequence
  Synthetic
FSVNYD

SEQ ID NO: 3 - protein, 4 aa, Artificial Sequence
  Synthetic
NVTV

SEQ ID NO: 4 - protein, 417 aa, Homo sapiens
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMLIPIAVGGALAGLVLIVLIAYLVGRKRSHAGYQTI